(19) United States
(12) Patent Application Publication
(10) Pub. No.: US 2007/0026458 A1
(43) Pub. Date: Feb. 1, 2007

(54) Assessing insulin resistance using biomarkers

(75) Inventors: David Polidori, Rancho Santa Fe, CA (US); Scott Siler, Hayward, CA (US); Leif Wennerberg, Mountain View, CA (US); Seth G. Michelson, San Jose, CA (US); Michael Reed, Menlo Park, CA (US)

Correspondence Address: ENTELOS, INC.;c/o FOLEY & LARDNER LLP, 1530 PAGE MILL RD., PALO ALTO, CA, 94304, US

(73) Assignee: Entelos, Inc.

(21) Appl. No.: 11/303,754

(22) Filed: Dec. 16, 2005

Related U.S. Application Data
(60) Provisional application No. 60/637,309, filed on Dec. 17, 2004.

(51) Int. Cl. G01N 33/53; G06F 19/00

(52) U.S. Cl. 435/7.1; 702/19

(57) ABSTRACT

The invention encompasses novel biomarkers and methods for assessing insulin resistance in a subject. The novel biomarkers of the invention include various plasma constituent (e.g., insulin, glucose, lactate and/or triglyceride) concentrations. The methods of the invention include measuring various plasma constituent concentrations and calculating a predicted euglycemic hyperinsulinemic clamp glucose infusion rate (GIR) based on the plasma constituent concentrations.

### CROSS-REFERENCE TO RELATED APPLICATIONS

This application claims the benefit of U.S. Provisional Application No. 60/637,309, filed Dec. 17, 2004, incorporated herein by reference.

### I. INTRODUCTION

A. Field of the Invention

This invention relates to novel biomarkers and methods of using the same for assessing insulin resistance in a subject.

B. Background of the Invention

Insulin resistance is a state in which physiologic concentrations of insulin produce a subnormal biologic response. In some cases, the abnormalities in how the body uses insulin lead to a compensatory increase in insulin secretion. Insulin resistance underlies abnormalities of glucose, lipid and blood pressure homeostasis. This cluster of metabolic abnormalities is referred to as insulin resistance syndrome, syndrome X, or the metabolic syndrome, and is related to type 2 diabetes, obesity, hypertension, and dyslipidemia. Insulin resistance also is directly related to the risk of developing atherosclerosis and cardiovascular disease. Typically, insulin resistance is present long before the clinical manifestation of the individual components of the syndrome.

Accurate measurement of insulin resistance in a clinical setting is not trivial, typically relying on combinations of oral or intravenous glucose and/or insulin combined with multiple blood samples (Ferrannini and Mari, J Hypertens. 16:895-906 (1998); Wallace and Matthews, Diabet. Med 19:527-534 (2002)). The standard for measuring insulin resistance, against which most measures are compared, is the euglycemic hyperinsulinemic clamp (DeFronzo, et al., Am J Physiol 237:E214-E223 (1979)).

Because this method is difficult and time consuming to perform, most clinicians use less complicated assessments to diagnose and monitor diabetes and insulin resistance. Typically, overnight fasting blood samples are analyzed with diagnostic kits. Occasionally, an oral glucose tolerance test (OGTT) may be performed, and some work has been done to develop insulin sensitivity measures from an OGTT (Matsuda and DeFronzo, Diabetes Care 22:1462-1470 (1999)). However, performing an OGTT is more inconvenient than fasting blood measures. In short, the characterization of any one pathophysiology and selection of an appropriate therapy in the clinical setting are generally less than optimal.

A biomarker correlated with insulin resistance as measured by an accepted benchmark would have clear utility at several stages of diabetes care and management: in selecting and adjusting therapies, in drug development, and in clinical and epidemiological research. Biomarkers for insulin sensitivity already have been used in lieu of more laborious clinical measures to interpret clinical data (Nagasaka, et al., Diabet. Med 21:136-141 (2004); U.K. Prospective Diabetes Study Group, Diabetes 44:1249-1258 (1995)). Much work has been done on finding measurements to predict insulin sensitivity. Wallace and Matthews (Diabet. Med 19:527-534 (2002)) and Radziuk (J Clin Endocrinol Metab 85:4426-4433 (2000)) provide useful reviews.

Current scientific dialog about insulin sensitivity biomarkers focuses on HOMA, which is simply proportional to the product of fasting insulin and glucose, and QUICKI, which is essentially the reciprocal of the log of HOMA (Matthews et al., Diabetologia 28:412-419 (1985); Katz et al., J Clin Endocrinol Metab 85:2402-2410 (2000)):

$$HOMA = \frac{[ fasting insulin ( uU \text{/} ml ) ] \times [ fasting glucose ( mg \text{/} dl ) ]}{405}$$

$$QUICKI = \frac{1}{\begin{matrix}log ( [ fasting insulin ( uU \text{/} ml ) ] \times \\ [ fasting glucose ( mg \text{/} dl ) ] )\end{matrix}}$$

Two recently published comparisons of HOMA and hyperinsulinemic-euglycemic clamp measurements (Bonora et al., Diabetes Care 23:57-63 (2000); Rabasa-Lhoret et al., J Clin Endocrinol Metab 88:4917-4923 (2003), and one study of QUICKI (Katz et al., 2000) emphasized correlations between the log of HOMA and insulin sensitivity as measured by euglycemic hyperinsulinemic clamp.

Quite good correlations between these markers and hyperinsulinemic euglycemic clamp results are found in some studies (Wallace, et al., Diabetes Care 27:1487-1495 (2004); Hermans, et al., Diabetologia 42:678-687 (1999)), particularly when a broad range of patients (from severe type 2 diabetics to normals) is included. However, in specific subpopulations—healthy, diabetic, or insulin-resistant—R² values rarely reach 50% (Katz, et al., J Clin Endocrinol Metab 85:2402-2410 (2000); Soonthompun, et al., J Clin Endocrinol Metab 88:1019-1023 (2003); Yokoyama, et al., Diabetes Care 26:2426-2432 (2003); Brun, et al., Diabetes Care 23:1037-1038 (2000); Matsuda and DeFronzo, Diabetes Care 22:1462-1470 (1999); Abbasi and Reaven, Metabolism 51:235-237 (2002); Kim, et al., Diabetes Care 27:1998-2002 (2004); Cutfield, et al., Pediatr. Diabetes 4:119-125 (2003); and Kuo, et al., Diabet. Med 19:735-740 (2002)). These values leave room for improvement. Compared to insulin alone, it is not clear that HOMA-IR or QUICKI are better predictors of insulin sensitivity. The difference in correlation of the standard HOMA and QUICKI measurements to the isoglycemic and euglycemic clamp measurements limits the value of these measures for diagnosing type 2 diabetes and assessing insulin resistance. Correlations between more rigorous clinical measures and hyperinsulinemic euglycemic clamp results, such as an oral glucose tolerance test, insulin suppression test, or an hyperinsulinemic isoglycemic clamp may not be much better (Katz, et al., J Clin Endocrinol Metab 85:2402-2410 (2000); Matsuda and DeFronzo, Diabetes Care 22:1462-1470 (1999); Stumvoll et al., Diabetes Care 23:295-301 (2000); Greenfield, et al., Diabetes 30:387-392 (1981)).

### SUMMARY OF THE INVENTION

One aspect of the invention provides biomarkers for assessing insulin resistance of a subject, said biomarker comprising a plasma insulin concentration, a plasma glucose concentration, and a plasma lactate concentration, wherein the subject fasts prior to measuring the plasma insulin, glucose and lactate concentrations. In a preferred embodiment the biomarker consists of a plasma insulin concentration, a plasma glucose concentration, and a plasma lactate concentration. Preferably the subject fasts for 12 to 24 hours prior to measuring the plasma insulin, glucose and lactate concentrations.

Another aspect of the invention provide biomarkers for assessing insulin resistance of a lactate-associated subject, said biomarker comprising a plasma insulin concentration and a plasma lactate concentration, wherein the subject fasts prior to measuring the plasma insulin and lactate concentrations. In a preferred embodiment, the biomarker consists of a plasma insulin concentration and a plasma lactate concentration.

Yet another aspect of the invention provides biomarkers for assessing insulin resistance of a glucose-associated subject, said biomarker comprising a plasma insulin concentration, a plasma glucose concentration, a plasma lactate concentration, and a plasma triglyceride concentration, wherein the subject fasts prior to measuring the plasma insulin, glucose, lactate and triglyceride concentrations. In a preferred embodiment, the biomarker consists of a plasma insulin concentration, a plasma glucose concentration, a plasma lactate concentration, and a plasma triglyceride concentration.

An aspect of the invention provides biomarkers for assessing insulin resistance of a subject, said biomarker comprising a plasma insulin concentration, a plasma glucose concentration, a plasma lactate concentration, a plasma HbA1c concentration, a plasma glycerol concentration, and a plasma C-peptide concentration, wherein the plasma insulin, glucose, lactate, HbA1c, glycerol and C-peptide concentrations are measured about two hours to about four hours after the subject consumers a heavy meal. In a preferred embodiment, the biomarker consists of a plasma insulin concentration, a plasma glucose concentration, a plasma lactate concentration, a plasma HbA1c concentration, a plasma glycerol concentration, and a plasma C-peptide concentration. One embodiment includes a biomarker having the formula:

$$GIR = 776 - 216 * plasma C - peptide - 14.6 * Hbalc - 0.05 plasma glycerol - 417 * plasma glycerol + 4.55 * plasma insulin + 1.80 * plasma lactate .$$

Typically a GIR value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is calculated as the rate of glucose infusion (mg/min) per lean body mass (kg-LBM).

Another aspect of the invention provides biomarkers for assessing insulin resistance of a subject comprising a plasma insulin concentration, a plasma glucose concentration, a plasma lactate concentration, a plasma glucagon concentration, a plasma free fatty acid concentration, plasma triglycerides concentration and a deviation of measured plasma glucose concentration from average plasma glucose concentration, wherein the plasma insulin, glucose, lactate, glucagon and free fatty acid concentrations are measured in the subject three hours after a heavy meal. In a preferred embodiment, the biomarker consists of a plasma insulin concentration, a plasma glucose concentration, a plasma lactate concentration, a plasma glucagon concentration, a plasma free fatty acid concentration, and a deviation of measured plasma glucose concentration from average plasma glucose concentration. One embodiment includes a biomarker having the formula:

$$GIR = 323 + 2.4 * plasmaFFA + 0.33 * plasma glucagon - 0.149 * plasma glucose - 2.46 * plasma insulin - 1.17 * plasma lactate + 0.092 * plasma TG + 0.503 * ( glucose deviation from avg glucose ) .$$

Typically a GIR value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is calculated as the rate of glucose infusion (mg/min) per lean body mass (kg-LBM).

One aspect of the invention provides methods of assessing insulin resistance of a subject comprising (a) measuring a plasma insulin concentration in the fasting subject; (b) measuring a plasma glucose concentration in the fasting subject; (c) measuring a plasma lactate concentration in the fasting subject; (d) calculating a predicted euglycemic hyperinsulinemic clamp glucose infusion rate (GIR); and (e) diagnosing the subject as being insulin resistant when the predicted GIR has a value of less than about 6 mg/kg-min. More preferably, a predicted GIR Value of less than about 5 mg/kg-min indicates insulin resistance in the subject. Most preferably a predicted GIR value of less than about 4 mg/kg-min indicates insulin resistance in the subject. In a preferred embodiment, the predicted GIR value is calculated as the rate of glucose infusion (mg/min) per lean body mass (kg-LBM).

One aspect of the invention provides methods of assessing insulin resistance of a lactate-associated fasting subject comprising (a) measuring a plasma insulin concentration in the lactate-associated fasting subject; (b) measuring a plasma lactate concentration in the lactate-associated fasting subject; (c) calculating a predicted euglycemic hyperinsulinemic clamp glucose infusion rate (GIR) and (d) diagnosing the subject as being insulin resistant when the predicted GIR has a value of less than about 6 mg/kg-min. More preferably, a predicted GIR value of less than about 5 mg/kg-min indicates insulin resistance in the subject. Most preferably a predicted GIR value of less than about 4 mg/kg-min indicates insulin resistance in the subject. In a preferred embodiment, the predicted GIR value is calculated as the rate of glucose infusion (mg/min) per lean body mass (kg-LBM).

Yet another aspect of the invention provides methods of assessing insulin resistance of a glucose-associated fasting subject comprising (a) measuring a plasma insulin concentration in the glucose-associated fasting subject; (b) measuring a plasma glucose concentration in the glucose-associated fasting subject; (c) measuring a plasma lactate concentration in the glucose-associated fasting subject; (d) measuring a plasma triglyceride concentration in the glucose-associated fasting subject; (e) calculating a predicted euglycemic hyperinsulinemic clamp glucose infusion rate (GIR); and (f) diagnosing a subject as being insulin resistant when the predicted GIR has a value of less than about 6 mg/kg-min. More preferably, a predicted GIR value of less than about 5 mg/kg-min indicates insulin resistance in the subject. Most preferably a predicted GIR value of less than 4 mg/kg-min indicates insulin resistance in the subject. In a preferred embodiment, the GIR value is calculated as the rate of glucose infusion (mg/min) per lean body mass (kg-LBM).

An aspect of the invention provides methods of assessing insulin resistance of a subject comprising (a) measuring a plasma insulin concentration in the subject about two to about four hours after a heavy meal; (b) measuring a plasma glucose concentration in the subject about two to about four hours after a heavy meal; (c) measuring a plasma lactate concentration in the subject about two to about four hours after a heavy meal; (d) measuring a plasma glycosylated hemoglobin (HbA1c) concentration about two to about four hours after a heavy meal; (e) measuring a plasma glycerol concentration in the subject about two to about four hours after a heavy meal; (f) measuring a plasma C-peptide concentration in the subject about two to about four hours after a heavy meal; (g) calculating a predicted hyperinsulinemic clamp glucose infusion rate (GIR) using the formula:

$$\begin{matrix}\begin{matrix}GIR = 776 - 216 * plasma C - peptide - \\ 14.6 * Hbalc - 0.05 plasma glucose -\end{matrix} \\ 417 * plasma glycerol + 4.55 * \\ plasma insulin + 180 * plasma lactate .\end{matrix} ; and$$

(h) assessing insulin resistance in the subject when the (GIR) is less than about 6 mg/kg-min. More preferably, a predicted GIR value of less than about 5 mg/kg-min indicates insulin resistance in the subject. Most preferably a predicted GIR value of less than about 4 mg/kg-min indicates insulin resistance in the subject. In a preferred embodiment, the predicted GIR value is calculated as the rate of glucose infusion (mg/min) per lean body mass (kg-LBM).

Yet another aspect of the invention provides methods of assessing insulin resistance of a subject comprising (a) measuring a plasma insulin concentration in the subject about three hours after a moderate meal; (b) measuring a plasma glucose concentration in the subject about three hours after a moderate meal; (c) measuring a plasma lactate concentration in the subject about three hours after a moderate meal; (d) measuring a plasma glucagon concentration about three hours after a moderate meal; (e) measuring a plasma free fatty acid concentration in the subject about three hours after a moderate meal; (f) measuring a deviation of measured plasma glucose concentration from average plasma glucose concentration in the subject about three hours after a moderate meal; (g) calculating a predicted euglycemic hyperinsulinemic clamp glucose infusion rate (GIR) using the formula:

$$\begin{matrix}GIR = 323 + 2.4 * plasmaFFA + 0.33 * plasma glucagon - \\ 0.149 * plasma glucose - 2.46 * plasma insulin - \\ 1.17 * plasma lactate + 0.092 * plasma TG + \\ 0.503 * ( glucose deviation from avg glucose ) .\end{matrix} ;$$

and (h) assessing insulin resistance in the subject when the predicted (GIR) is less than about 6 mg/kg-min. More preferably, a predicted GIR value of less than about 5 mg/kg-min indicates insulin resistance in the subject. Most preferably a predicted GIR value of less than about 4 mg/kg-min indicates insulin resistance in the subject. In a preferred embodiment, the predicted GIR value is calculated as the rate of glucose infusion (mg/min) per lean body mass (kg-LBM).

Another aspect of the invention provides kits for practicing the methods of the invention. In one implementation the kit comprises a device for obtaining a blood sample from the subject, a reagent for measuring a concentration of glucose (G) in the blood sample, a reagent for measuring a concentration of lactate (L) in the blood sample, a reagent for measuring a concentration of insulin (I) in the blood sample, and instructions for use. Alternatively, the kit can comprise a device for obtaining a blood sample from the subject, a reagent for measuring a concentration of glycosylated hemoglobin (HbA 1 c) in the blood sample, a reagent for measuring a concentration of lactate (L) in the blood sample, a reagent for measuring a concentration of insulin (I) in the blood sample, and instructions for use.

It will be appreciated by one of skill in the art that the embodiments summarized above may be used together in any suitable combination to generate additional embodiments not expressly recited above, and that such embodiments are considered to be part of the present invention.

### II. BRIEF DESCRIPTION OF THE FIGURES

FIG. 1 illustrates biomarker predictions of euglycemic hyperinsulinemic clamp glucose infusion rate (GIR) values based on an optimal fasting biomarker of the invention and on fasting insulin only. The diameters of the symbols correspond to the prevalence weightings of the individual virtual patients. Black symbols correspond to the optimal fasting biomarker, with an R² of 59%; gray symbols correspond to insulin alone, with an R² of 45%.

FIGS. 2A and 2B illustrate a Bivariate Normal distribution with means and standard deviations of the two variates equal to the mean and standard deviation of the GIR values observed in the virtual patient population, computed using prevalence weightings. The correlation of the two variables is indicated by the trend of the distribution along the diagonal. R² was assumed to match the 59% value of the optimal fasting biomarker.

FIGS. 3A and 3B illustrate a Bivariate Normal distribution with means and standard deviations of the two variates equal to the mean and standard deviation of the GIR values observed in the virtual patient population, computed using the prevalence weightings. The lack of correlation of the two variables is indicated by the circular distribution, corresponding to an R² of zero.

FIG. 4 provides points from theoretical or simulated receiver operated characteristic (ROC) curves for various thresholds and biomarker values for the two prevalence distributions in FIG. 2 and FIG. 3. The straight line corresponds to zero correlation, the curve to R²=59%.

FIG. 5 illustrates the distance of points in FIG. 4 from the upper right corner of the graph (sensitivity=1, 1-specificity=0), as a function of threshold. The dashed curve corresponds to an uncorrelated biomarker and the solid curve to an idealized R² of 59%.

FIGS. 6A and 6B illustrate plasma glucose and insulin, respectively, in type 2 diabetic virtual patients in response to a twenty-four-hour (1440 minute) fast.

FIGS. 7A and 7B illustrate plasma glucose and insulin, respectively, in type 2 diabetic virtual patients in response to three standard mixed meals over twenty-four hours.

FIG. 8 illustrates plasma glucose in type 2 diabetic virtual patients in response to an oral glucose load (75 g) administered at sixty minutes.

FIGS. 9A and 9B illustrate muscle glucose uptake and plasma glucose, respectively, vs. plasma insulin concentration in type 2 diabetic virtual patients in response to an oral glucose load (75 g). Data was extracted from zero to one hundred fifty minutes of the oral glucose tolerance test (OGTT).

FIGS. 10A and 10B illustrate plasma glucose and insulin, respectively, in ten type 2 diabetic virtual patients in response to an intravenous glucose bolus (0.3 mg/kg) administered at sixty minutes.

FIGS. 11A and 11B illustrate plasma insulin and glucose, respectively, in type 2 diabetic virtual patients in response to a hyperinsulinemic euglycemic clamp. Insulin infusion started at zero minutes, with glucose infusion employed as needed to maintain euglycemia.

FIG. 12 illustrates (A) glucose infusion rate, (B) muscle glucose uptake, (C) hepatic glucose output, and (D) total lipolysis rate in type 2 diabetic virtual patients in response to a hyperinsulinemic euglycemic clamp. Insulin infusion started at zero minutes, with glucose infusion employed as needed to maintain euglycemia.

FIGS. 13A and 13B illustrate plasma glucose and insulin, respectively, in type 2 diabetic virtual patients in response to a hyperglycemic clamp. Glucose infusion was initiated at sixty minutes.

FIG. 14 illustrates fasting plasma glucose and insulin values for all virtual type 2 diabetics in the Metabolism PhysioLab platform (diamonds), and those used in this analysis (squares). Virtual patients were excluded from the analysis if glucose pump activity did not turn on in simulated hyperinsulinemic euglycemic clamps when insulin was clamped at 60 mU/ml.

FIG. 15 illustrates the relationship between HOMA and glucose disposal for type 2 diabetics reported by Bonora et al. (2000). The line at 1 n HOMA=1 corresponds to a HOMA of ˜2.7. Most of the virtual patients have values above the line (FIG. 8). R² are shown for the whole data set and for the diabetics corresponding to the virtual patients in this study.

FIG. 16 illustrates the distribution of HOMA scores for virtual patients used in this study.

FIG. 17 illustrates a comparison of QUICKI and insulin sensitivity as measured by a hyperinsulinemic-isoglycemic clamp for human type 2 diabetics (black squares) and the virtual patients (open diamonds) used in this study. Insulin sensitivity data is from Katz et al. (2000): the clamp glucose infusion rate is “SI_{clamp}”, which is a normalization of the glucose infusion rate by body weight, baseline glucose, and the change in insulin level from the baseline during the clamp.

FIG. 18 illustrates the prevalence weighting and corresponding least-squares line through the virtual patient data that yielded an R² of 48% and a slope and intercept within the 90% error bars of the line through Katz et al.'s data. The dotted lines show the profile of the normal distribution of prevalences over a width of two standard deviations on each side of the line.

FIG. 19 shows the distribution of weightings among the patients as a function of their fasting glucose and insulin values. FIG. 19 illustrates fasting glucose and insulin values of virtual patients, along with prevalence weightings. The top two-thirds of the weightings are indicated by open circles with diameters proportional to the relative weightings. The remaining prevalence-weighted virtual patients are represented as solid circle of fixed diameter (unrelated to relative weighting).

FIG. 20 illustrates the correlation between isoglycemic and euglycemic clamp measures used in this study (R²=45%). The prevalence weighting was based on isoglycemic clamp simulations, and then used when determining correlations with euglycemic clamp data.

FIG. 21 illustrates simulated GIR versus fitted function of various biomarker variables. (A) Weighted correlation between insulin alone as a predictor of insulin sensitivity and GIR: R²=45%. (B) Weighted correlation between a linear combination of insulin and lactate as a predictor of insulin sensitivity and GIR: R²=52%. (C) Weighted correlation between a linear combination of insulin and HbA1c as a predictor of insulin sensitivity and GIR: R²=51%. (D) Weighted correlation between a linear combination of insulin, lactate, and HbA1c as a predictor of insulin resistance and GIR: R²=59%.

FIG. 22 shows changes in weighted residuals for each patient in the step-wise regression compared to fitting with insulin alone. Prevalence weightings are also shown. When sorted by fit to the final regression line, the effects of regressing on insulin and lactate or insulin and HbA1c. Note inverted scale for errors and log scale for prevalence weightings.

FIG. 23 illustrates the subpopulation-specific biomarkers for (A) “lactate-associated” and (B) “glucose-associated” insulin resistance. The lactate-associated population marker relies on just two plasma components: insulin and lactate, with an R² of 62%. The glucose-associated population can be well characterized by a more complex biomarker composed of insulin, triglycerides, lactate, and glucose, with an R² of 79%. Without insulin, the marker's correlation decreases slightly, to R²=62%.

FIG. 24 shows ROC points for HOMA, QUICKI, fasting insulin and the optimal fasting biomarker of the invention.

FIG. 25 illustrates the distance of points in FIG. 23 from the upper left corner of the graph (sensitivity=1, 1-specificity=0), as a function of threshold. The biomarkers corresponding to each line are indicated. Two idealized curves are also shown for comparison.

FIG. 26 shows an example of delay times and frequency with which they were sampled for a specific simulation.

FIG. 27 shows the best-case multivariate correlations between postprandial plasma quantities and insulin sensitivity for light (FIG. 27A) and heavy (FIG. 27B) meals (red). The optimal fasting biomarker results (black) are shown for comparison.

FIG. 28 shows R² values for the ten random perturbations of sample times around the two-hour time point for the light test meal (FIG. 28A) and heavy test meal (FIG. 28B).

FIG. 29 illustrates ROC points for all ten sets of random perturbations to the two-hour sample time for the heavy meal. Colored symbols correspond to different perturbation sets. The black symbols correspond to the optimal fasting biomarker. Thresholds are in the range of values shown in FIG. 27.

FIG. 30 illustrates the threshold dependence of sensitivities and specificities at two hours after a heavy meal. Plot shows the distance of points in FIG. 29 from the upper left corner of the graph (sensitivity=1, 1-specificity=0) as a function of threshold. The square symbols represent the average values of the ten sets of perturbations to the sample times, and the error bars represent two standard deviations. The round symbols represent the optimal fasting biomarker. The dashed line represents the idealized case for R²=75%, which is the maximal value from the heavy meal biomarker fits.

FIG. 31 illustrates the threshold dependence of sensitivities and specificities at three hours after a light meal. The plot shows the distance of ROC points from the upper left corner of the graph (sensitivity=1, 1-specificity=0) as a function of threshold. The square symbols represent the average values of the ten sets of perturbations to the sample times, and the error bars represent two standard deviations. The round symbols represent the optimal fasting biomarker. The dashed line represents the idealized case for R²=70%, which is approximately the average value from the light meal biomarker fits.

FIG. 32 illustrates the threshold dependence of sensitivities and specificities at three hours after a moderate meal. Plot shows the distance of points from the upper left corner of the graph (sensitivity=1, 1-specificity=0), as a function of threshold. The square symbol represents the average values of the ten sets of perturbations to the sample times, and the error bars represent two standard deviations. The round symbols represent the optimal fasting biomarker. The dashed line represents the idealized case for R²=80%, which is approximately the average from the moderate meal biomarker fits.

FIG. 33 shows the threshold dependence of sensitivities and specificities, using six regressors, at two hours after a heavy meal. Plot shows the distance of points from the upper left corner of the graph (sensitivity=1, 1-specificity=0), as a function of threshold. The square symbols represent the average values of the ten sets of perturbations to the sample times, and the error bars represent two standard deviations. The round symbols represent the optimal fasting biomarker. The dashed line represents the idealized case for R²=75%, which is approximately the average from the heavy meal biomarker fits.

FIG. 34 shows the threshold dependence of sensitivities and specificities, using seven regressors, at three hours after a moderate meal. Plot shows the distance of points from the upper left corner of the graph (sensitivity=1, 1-specificity=0), as a function of threshold. The square symbols represent the average values of the ten sets of perturbations to the sample times, and the error bars represent two standard deviations. The round symbols represent the optimal fasting biomarker. The dashed line represents the idealized case for R²=80%, which is approximately the average from the moderate meal biomarker fits.

### III. DETAILED DESCRIPTION

A. Overview

The invention encompasses novel biomarkers and methods for assessing insulin resistance in a subject. The novel biomarkers of the invention include various plasma constituent (e.g., insulin, glucose, lactate and/or triglyceride) concentrations. The methods of the invention include measuring various plasma constituent concentrations and calculating a predicted euglycemic hyperinsulinemic clamp glucose infusion rate (GIR) based on the plasma constituent concentrations.

B. Definitions

A “biomarker,” as used herein, is a (set of) biological characteristic(s) that can be objectively measured and used to infer another quantity of interest, such as a biological process or a response to an intervention.

As used herein, the term “subject” refers to a real individual, preferably to a human. Whereas, the term “virtual patient” refers to mathematical representations of a subject in a computer model of macronutrient metabolism.

As used herein, the terms “insulin resistance” and “insulin resistant” refer to a state in which the body has a reduced response to the action of insulin hormone although enough insulin is produced.

C. Virtual Patients

Biosimulation has the potential to improve the utility and value of diagnostic kits in determining insulin resistance. A computer model of human multiple macronutrient metabolism and diabetes related disorders was initially developed using a representation of normal physiology, substantially in the manner described in U.S. Patent Application Publication 2003-0058245 A1, incorporated herein by reference. A normal virtual patient was created using parameter sets, each of which mathematically describes a relationship between physiological variables relevant to metabolism. For example, the parameter set for liver glycogenolysis describes the relationships between glycogenolysis rate and plasma glucose, insulin, glucagon, and epinephrine. Each physiological relationship is calibrated using empirical data, with the overall behavior of the normal virtual patient (who is the sum of many parameter sets) validated using experimental protocols that represent complex behavior such as the response to mixed meal feeding.

Once a normal physiology has been defined, specific defects, e.g., those related to the pathophysiology of diabetes, in the normal physiology can be modeled and simulated. The term “defect” as used herein means an imperfection, failure, or absence of a biological variable or a biological process associated with a disease state. Diabetes, including type 2 diabetes, is a disease resulting from a heterogeneous combination of defects. The computer model can be designed so that a user can simulate defects of varying severity, in isolation or combination, in order to create various diabetic and prediabetic patient types. The model thus can provide several virtual patient types of varying degrees of diabetes.

Type 2 diabetic virtual patients are created by manipulating each parameter set in the normal subject to describe the changes in relationships between physiological variables that occur with diabetes. For example, the dose response curve for the effect of insulin on muscle glucose uptake may be altered to represent reduced insulin sensitivity. Each virtual patient is then validated in a variety of experimental protocols to confirm that its behavior is consistent with reported human clinical data. For example, the diabetic virtual patient may have reduced glucose uptake and elevated hepatic glucose output in a hyperinsulinemic euglycemic clamp when compared to the normal patient, but the magnitude of these changes must be within reported ranges.

The computer model of virtual patients can be configured so as to compute many outputs including: biological variables like plasma glucose, insulin, C-peptide, FFA, triglycerides, lactate, glycerol, amino acids, glucagon, epinephrine, muscle glycogen, liver glycogen; body weight and body mass index; respiratory quotient and other measures of substrate utilization; clinical indices of long-term hyperglycemia including glycosylated hemoglobin (% HbA1c) and fructosamine; substrate and energy balances; as well as metabolic fluxes including muscle glucose uptake, hepatic glucose output, glucose disposal rate, lipolysis rate, glycogen synthesis, and glycogenolysis rates. The outputs can also be presented in several commonly used units.

Parameters can also be used to specify stimuli and environmental factors as well as intrinsic biological properties. In addition, the computer model can simulate in vivo experimental protocols including: pancreatic clamps; infusions of glucose, insulin, glucagon, somatostatin, and free fatty acid (FFA); intravenous glucose tolerance test (IVGTT); oral glucose tolerance test (OGTT); and insulin secretion experiments demonstrating acute and steady state insulin response to plasma glucose steps. Furthermore, model parameters can be chosen to represent various environmental changes such as diets with different nutrient compositions, as well as various levels of physical activity and exercise.

The computer model was designed to be completely observable, meaning that every entity represented in the platform can be sampled continuously during the course of an experiment. For example, one is able to measure plasma, portal, hepatic, sinusoidal, and intracellular glucose and insulin concentrations during many different types of experiments.

The responses of the ten virtual patients to these experimental protocols were diverse, reflecting the diversity of real type 2 diabetic patients. Extensive virtual patient profiles that include both clinically observable and less observable measurements that can shed light on the underlying patient pathophysiology were generated.

D. Biomarker of Insulin Resistance in Fasting Subjects

One aspect of the invention provides biomarkers for assessing insulin resistance in a subject, said biomarker comprising a plasma insulin concentration, a plasma glucose concentration and a plasma lactate concentration; wherein the subject fasts prior to measurement of the plasma insulin, glucose and lactate concentrations. As used herein, the term “fast” or “fasting” refers to abstaining from food. Preferably the subject fasts for eight hours, more preferably at least ten hours, most preferably at least twelve hour prior to measurement of plasma concentrations. In addition, it is preferred that the subject fasts for no longer than sixteen hours. In a preferred embodiment the biomarker consists of a plasma insulin concentration, a plasma glucose concentration, and a plasma lactate concentration.

Biomarkers are useful for understanding the systemic complexities of a disease that are not readily measurable. The selection and interpretation of biomarkers is dependent on the relationship between the biomarker and the quantity of interest. In addition, a biomarker's predictive value depends on the conditions (experimental protocol, measurement time) under which it is measured. The present invention characterizes in detail a series of type 2 diabetic virtual patients and identifies optimal sets of single point plasma diagnostic tests under different test conditions. Each set of single point plasma diagnostic tests together are a biomarker for insulin resistance.

The computer model was used to identify three fasting plasma substances that have potential as a biomarker profile for insulin resistance: insulin, lactate, and HbA1c (or glucose). Regression analysis of these three values provides the biomarker equation.
GIR=100−4.74I+12.5L+10.2HbA1c
wherein I represents plasma insulin concentration, L represents plasma lactate concentration and HbA1c represents plasma glycosylated hemoglobin concentration. Plasma glycosylated hemoglobin is surrogate for plasma glucose concentration. Therefore, an alternative regression analysis provides a biomarker having the formula
GIR=126−5.05I+13.3L+0.370G
wherein I represents plasma insulin concentration, L represents plasma lactate concentration and G represents plasma glucose concentration. Typically a GIR value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is calculated as the rate of glucose infusion (mg/min) per lean body mass (kg-LBM).

A residuals analysis of the regression that defined the whole-population biomarker identified two subpopulations of virtual patients with apparently distinctive insulin resistances: “lactate-associated” and “glucose-associated.” The biomarkers specific for these subpopulations had quite high R² values, especially the glucose-correlated group compared to any previous literature reports. For the “lactate-associated” subjects, i.e., those subjects for whom inclusion of lactate improved the fit had less improvement when Hb1Ac was added to the regression fit, the optimal fasting biomarker consists of insulin and lactate alone, with a correlation of R²=62%.

The invention provides biomarkers for assessing insulin resistance in a lactate-associated subject comprising a plasma insulin concentration and a plasma lactate concentration, wherein the plasma insulin and lactate concentrations are measured in a fasting lactate-associated subject. In a preferred embodiment, the biomarker consists of a plasma insulin concentration and a plasma lactate concentration. In a preferred embodiment, the biomarker for assessing insulin resistance in a lactate-associated subject is:
GIR=114.0−5.88I+23.4L
wherein I represents plasma insulin concentration and L represents plasma lactate concentration. A GIR value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is calculated as the rate of glucose infusion (mg/min) per lean body mass (kg-LBM).

For the “glucose-associated” subjects, i.e., those subjects for whom inclusion of glucose improved the fit but had less improvement when lactate was added to the regression fit, the optimal fasting biomarker consists of insulin, lactate, glucose and triglyceride, with a correlation of R²=74%. Yet another aspect of the invention provides biomarkers for assessing insulin resistance in a glucose-associated subject comprising a plasma insulin concentration, a plasma glucose concentration, a plasma lactate concentration, and a plasma triglyceride concentration, wherein the plasma insulin, glucose, lactate and triglyceride concentrations are measured in a fasting glucose-associated subject. In a preferred embodiment, the biomarker consists of a plasma insulin concentration, a plasma glucose concentration, a plasma lactate concentration, and a plasma triglyceride concentration. Preferably the biomarker is
GIR=−12.6+0.82G+16.13L+0.076TG−3.42I
wherein G represents plasma glucose concentration, L represents plasma lactate concentration, TG represents plasma triglyceride concentration and I represents plasma insulin concentration. Typically a GIR value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is calculated as the rate of glucose infusion (mg/min) per lean body mass (kg-LBM).

E. Postprandial Biomarker of Insulin Resistance

An aspect of the invention provides biomarkers for assessing insulin resistance in a subject comprising a plasma insulin concentration, a plasma glucose concentration, a plasma lactate concentration, a plasma HbA1c concentration, a plasma glycerol concentration, and a plasma C-peptide concentration, wherein the plasma insulin, glucose, lactate, HbA1c, glycerol and C-peptide concentrations are measured about two hours to about four hours after the subject consumes a heavy meal. In a preferred embodiment, the biomarker consists of a plasma insulin concentration, a plasma glucose concentration, a plasma lactate concentration, a plasma HbA1c concentration, a plasma glycerol concentration, and a plasma C-peptide concentration.

Another aspect of the invention provides biomarkers for assessing insulin resistance in a subject comprising a plasma insulin concentration, a plasma glucose concentration, a plasma lactate concentration, a plasma glucagon concentration, a plasma free fatty acid concentration, a plasma triglyceride concentration and a deviation of measured plasma glucose concentration from average plasma glucose concentration, wherein the plasma insulin, glucose, lactate, glucagon and free fatty acid concentrations are measured about three hours after the subject consumes a heavy meal. In a preferred embodiment, the biomarker consists of a plasma insulin concentration, a plasma glucose concentration, a plasma lactate concentration, a plasma glucagon concentration, a plasma free fatty acid concentration, a plasma triglyceride concentration and a deviation of measured plasma glucose concentration from average plasma glucose concentration.

Due to difficulties in obtaining subject compliance with a 24-hour fast, as used in simulating the fasting biomarkers discussed above, correlations between postprandial (after meal) plasma values and insulin resistance were investigated. Thus correlations between plasma quantities measured throughout a three-meal day and the following night were studied. Preliminary results of that analysis suggest that for the whole virtual patient population, quite high prevalence-weighted correlations with insulin sensitivity, as measured by euglycemic hyperinsulinemic clamp, were observed, e.g., 80% for insulin a few hours after the evening meal, suggesting that a meal challenge protocol could enhance the ultimate R² for the biomarker.

While the optimal fasting biomarker described above is considerably better than those for HOMA and QUICKI, postprandial biomarkers of the invention have advantages over the optimal fasting biomarker as evidenced by a higher correlation with insulin sensitivity (R²>70% vs. 59%, respectively) and greater sensitivity and specificity to the following measures. The inventors have identified several separate postprandial biomarker profiles. The first biomarker profile for a heavy meal at a two-hour postprandial sampling time consists of plasma C-peptide, HbA1c, glycerol, insulin, and lactate. The second biomarker profile for moderate meal at a three-hour postprandial sampling time consists of plasma free fatty acid (FFA,) glucagon, glucose, insulin, lactate, triglyceride (TG), and deviation of HbA1c from average glucose.

Additionally, for a single time-point postprandial biomarker to be effective, the meal should contain at least 750 calories and the sampling time should be somewhere between two to four hours after the meal. In the same way as the fasting biomarker discussed above, insulin is the most important regressor and lactate also played an important role.

Only plasma quantities that varied by more than 5% were considered relevant, i.e., eleven relevant factors out of twenty nine. The multivariate correlation of all remaining regressors was calculated to determine the best possible R², and ROC points were established as above. From this group of eleven, a core set of regressors (i.e., those that contributed the most to the ultimate predictability of the biomarker) was determined by systematically removing those that yielded the lowest predictive value. The final biomarker is termed “the most efficient biomarker.” For the two cases that showed the greatest difference from a fasting measure (heavy meal, two-hour time point; and moderate meal, three-hour time point), the most efficient biomarkers were:

$$\begin{matrix}GIR = 776 - 216 * plasma C - peptide - \\ 14.6 * Hbalc - 0.05 plasma glucose - \\ 417 * plasma glycerol + 4.55 * plasma insulin + \\ 1.80 * plasma lactate\end{matrix}$$

$$and$$

$$\begin{matrix}GIR = 323 + 2.4 * plasmaFFA + 0.33 * plasma glucagon - \\ 0.149 * plasma glucose - 2.46 * plasma insulin - \\ 1.17 * plasma lactate + 0.092 * plasma TG + \\ 0.503 * ( glucose deviation from avg glucose ) .\end{matrix}$$

respectively. The last term for the moderate meal, “glucose deviation from avg glucose,” is a measure of the difference between the glucose level at three hours and the weighted average of glucose levels over thirty and ninety days, where the weightings are 0.3 and 0.7 respectively. A linear function of this weighted average is equal to HbA1c in the model. The weighted average glucose is a dimensionally appropriate proxy for HbA1c, and can be computed from HbA1c measures from the following:
HbA1c=0.0281*avg glucose+2.17
(see Rohlfing et al. 2002).

As described above, plasma insulin, glucose and lactate concentrations can be determined by any method. Similarly, the plasma concentration of free fatty acid glucagon, triglyceride, glycosylated hemoglobin or C-peptide can be measured using any method known to one of skill in the art.

F. Methods of Assessing Insulin Resistance

One aspect of the invention provides methods of assessing insulin resistance in a subject comprising (a) measuring a plasma insulin concentration in the fasting subject; (b) measuring a plasma glucose concentration in the fasting subject; (c) measuring a plasma lactate concentration in the subject; (d) calculating a predicted GIR; and (e) diagnosing the subject as being insulin resistant when the predicted GIR has a value of less than about 6 mg/kg-min. More preferably, a predicted GIR value of less than about 5 mg/kg-min indicates insulin resistance in the subject. Most preferably predicted a GIR value of less than about 4 mg/kg-min indicates insulin resistance in the subject. In a preferred embodiment, the predicted GIR value is calculated as the rate of glucose infusion (mg/min) per lean body mass (kg-LBM).

Another aspect of the invention provides methods of assessing insulin resistance in a lactate-associated fasting subject comprising (a) measuring a plasma insulin concentration in the lactate-associated fasting subject; (b) measuring a plasma glucose concentration in the lactate-associated fasting subject; (c) measuring a plasma lactate concentration in the lactate-associated fasting subject; (d) calculating a predicted GIR; and (e) diagnosing the subject as insulin resistant when the predicted GIR has a value of less than about 6 mg/kg-min. More preferably, a predicted GIR value of less than about 5 mg/kg-min indicates insulin resistance in the subject. Most preferably a predicted GIR value of less than about 4 mg/kg-min predicts insulin resistance in the subject. In a preferred embodiment, the predicted GIR value is calculated as the rate of glucose infusion (mg/min) per lean body mass (kg-LBM).

Yet another aspect of the invention provides methods of assessing insulin resistance in a glucose-associated fasting subject comprising (a) measuring a plasma insulin concentration in the glucose-associated fasting subject; (b) measuring a plasma glucose concentration in the glucose-associated fasting subject; (c) measuring a plasma lactate concentration in the glucose-associated fasting subject; (d) measuring a plasma triglyceride concentration in the glucose-associated fasting subject; (e) calculating a predicted GIR; and predicting (f) diagnosing the subject as insulin resistant when the predicted GIR has a value of less than about 6 mg/kg-min. More preferably, a predicted GIR value of less than about 5 mg/kg-min indicates insulin resistance in the subject. Most preferably a predicted GIR value of less than about 4 mg/kg-min predicts insulin resistance in the subject. In a preferred embodiment, the predicted GIR value is calculated as the rate of glucose infusion (mg/min) per lean body mass (kg-LBM).

An aspect of the invention provides methods of assessing insulin resistance in a subject comprising (a) measuring a plasma insulin concentration in the subject about two to about four hours after a heavy meal; (b) measuring a plasma glucose concentration in the subject about two to about four hours after a heavy meal; (c) measuring a plasma lactate concentration in the subject about two to about four hours after a heavy meal; (d) measuring a plasma glycosylated hemoglobin (HbA1c) concentration about two to about three hours after a heavy meal; (e) measuring a plasma glycerol concentration in the subject about two to about four hours after a heavy meal; (f) measuring a plasma C-peptide concentration in the subject about two to about four hours after a heavy meal; (g) calculating a predicted GIR using the formula:

$$\begin{matrix}\begin{matrix}GIR = 776 - 216 * plasma C - peptide - \\ 14.6 * Hbalc - 0.05 plasma glucose -\end{matrix} \\ 417 * plasma glycerol + 4.55 * \\ plasma insulin + plasma lactate\end{matrix} ;$$

and (h) diagnosing the subject as insulin resistant when the predicted GIR has a value of less than about 6 mg/kg-min. More preferably, a predicted GIR value of less than about 5 mg/kg-min indicates insulin resistance in the subject. Most preferably a predicted GIR value of less than 4 mg/kg-min indicates insulin resistance in the subject. In a preferred embodiment, the predicted GIR value is calculated as the rate of glucose infusion (mg/min) per lean body mass (kg-LBM).

Yet another aspect of the invention provides methods of assessing insulin resistance in a subject comprising (a) measuring a plasma insulin concentration in the subject about three hours after a moderate meal; (b) measuring a plasma glucose concentration in the subject about three hours after a moderate meal; (c) measuring a plasma lactate concentration in the subject about three hours after a moderate meal; (d) measuring a plasma glucagon concentration about three hours after a moderate meal; (e) measuring a plasma free fatty acid concentration in the subject about three hours after a moderate meal; (f) measuring a plasma triglyceride concentration in a subject about 3 hours after a moderate meal; (g) measuring deviation of measured plasma glucose concentration from average plasma glucose concentration in the subject about three hours after a moderate meal; (h) calculating a predicted glucose infusion rate (GIR) using the formula:

$$\begin{matrix}GIR = 323 + 2.4 * plasmaFFA + 0.33 * plasma glucagon - \\ 0.149 * plasma glucose - 2.46 * plasma insulin - \\ 1.17 * plasma lactate + 0.092 * plasma TG + \\ 0.503 * ( glucose deviation from avg glucose ) .\end{matrix} ;$$

and (i) assessing insulin resistance in the subject when the predicted GIR has a value of less than about 6 mg/kg-min is indicative of insulin resistance. More preferably, a GIR value of less than about 5 mg/kg-min predicts insulin resistance in the subject. Most preferably a GIR value of less than 4 mg/kg-min predicts insulin resistance in the subject. In a preferred embodiment, the GIR value is measured as the rate of glucose infusion (mg/min) per lean body mass (kg-LBM).

The methods of the invention can be practiced by a medical practitioner or by the subject. Plasma concentrations can be measured using any method or apparatus known to one of skill in the art. Preferably the methods will be practiced utilizing commercially available monitoring kits, however, the invention is not so limited.

The present invention also provides kits for performing the methods of the invention. Such kits can be prepared from readily available materials and reagents and can come in a variety of embodiments. For example, such kits can comprise, in an amount sufficient for at least one evaluation, any one or more of the following materials: test strips, devices for obtaining a blood sample, devices for piercing skin, vessels, sterilized buffers (e.g., phosphate buffered saline) or water, other reagents necessary or helpful to perform the method, and instructions. Typically, instructions include a tangible expression describing reagent concentration or at least one method parameter, such as the amount of reagent to be used, maintenance time periods for reagents, and the like, to allow the user to carry out the methods described above. Further the instruction can include charts, comparators, graphs or formulas for calculating the effective glucose infusion rate (GIR) as a measure of insulin resistance. In a preferred embodiment of the invention, a kit comprises a device for obtaining a blood sample from the subject, a reagent for measuring a concentration of glucose (G) in the blood sample, a reagent for measuring a concentration of lactate (L) in the blood sample, a reagent for measuring a concentration of insulin (I) in the blood sample, and instructions for use. In an alternative implementation, the kit comprises a device for obtaining a blood sample from the subject, a reagent for measuring a concentration of glycosylated hemoglobin (HbA1c) in the blood sample, a reagent for measuring a concentration of lactate (L) in the blood sample, a reagent for measuring a concentration of insulin (I) in the blood sample, and instructions for use. Plasma insulin, glucose and lactate concentrations can be determined by any method now known or later developed by those of skill in the art. There are, for example, the instruments described in U.S. Patents: U.S. Pat. Nos. 3,770,607; 3,838,033; 3,902,970; 3,925,183; 3,937,615; 4,005,002; 4,040,908; 4,086,631; 4,123,701; 4,127,448; 4,214,968; 4,217,196; 4,224,125; 4,225,410; 4,230,537; 4,260,680; 4,263,343; 4,265,250; 4,273,134; 4,301,412; 4,303,887; 4,366,033; 4,407,959; 4,413,628; 4,420,564; 4,431,004; 4,436,094; 4,440,175; 4,477,314; 4,477,575; 4,499,423; 4,517,291; 4,654,197; 4,671,288; 4,679,562; 4,682,602; 4,703,756; 4,711,245; 4,734,184; 4,750,496; 4,759,828; 4,789,804; 4,795,542; 4,805,624; 4,816,224; 4,820,399; 4,897,162; 4,897,173; 4,919,770; 4,927,516; 4,935,106; 4,938,860; 4,940,945; 4,970,145; 4,975,647; 4,999,582; 4,999,632; 5,108,564; 5,120,420; 5,128,015; 5,141,868; 5,192,415; 5,243,516; 5,264,103; 5,269,891; 5,288,636; 5,312,762; 5,352,351; 5,385,846; 5,395,504; 5,437,999; 5,469,846; 5,508,171; 5,508,203; 5,509,410; and 5,575,895; German Patent Specification 3,228,542; European Patent Specifications: 206,218; 230,472; 241,309; 255,291 and 471,986: and Japanese Published Patent Applications JP 63-128,252 and 63-111,453. There are also the methods and apparatus described in: Talbott, et al, “A New Microchemical Approach to Amperometric Analysis,” Microchemical Journal, Vol. 37, pp. 5-12 (1988); Morris, et al, “An Electrochemical Capillary Fill Device for the Analysis of Glucose Incorporating Glucose Oxidase and Ruthenium (III) Hexamine as Mediator, Electroanalysis,” Vol. 4, pp. 1-9 (1992); Cass, et al, “Ferrocene-Mediated Enzyme Electrode for Amperometric Determination of Glucose,” Anal. Chem., Vol. 56, pp. 667-671 (1984); Zhao, “Contributions of Suspending Medium to Electrical Impedance of Blood,” Biochimica et Biophysica Acta, Vol. 1201, pp. 179-185 (1994); Zhao, “Electrical Impedance and Haematocrit of Human Blood with Various Anticoagulants,” Physiol. Meas., Vol. 14, pp. 299-307 (1993); Muller, et al., “Influence of Hematocrit and Platelet Count on Impedance and Reactivity of Whole Blood for Electrical Aggregometry,” Journal of Pharmacological and Toxicological Methods, Vol. 34, pp. 17-22 (1995); Preidel, et al, “In Vitro Measurements with Electrocatalytic Glucose Sensor in Blood,” Biomed. Biochim. Acta, Vol. 48, pp. 897-903 (1989); Preidel, et al, “Glucose Measurements by Electrocatalytic Sensor in the Extracorporeal Blood Circulation of a Sheep,” Sensors and Actuators B, Vol. 2, pp. 257-263 (1990); Saeger, et al, “Influence of Urea on the Glucose Measurement by Electrocatalytic Sensor in the Extracorporeal Blood Circulation of a Sheep,” Biomed. Biochim. Acta, Vol. 50, pp. 885-891 (1991); Kasapbasioglu, et al, “An Impedance Based Ultra-Thin Platinum Island Film Glucose Sensor,” Sensors and Actuators B, Vol. 13-14, pp. 749-751 (1993); Beyer, et al, “Development and Application of a New Enzyme Sensor Type Based on the EIS-Capacitance Structure for Bioprocess Control,” Biosensors & Bioelectronics, Vol. 9, pp. 17-21 (1994); Mohri, et al, “Characteristic Response of Electrochemical Nonlinearity to Taste Compounds with a Gold Electrode Modified with 4-Aminobenzenethiol,” Bull. Chem. Soc. Jpn., Vol. 66, pp. 1328-1332 (1993); Cardosi, et al, “The Realization of Electron Transfer from Biological Molecules to Electrodes,” Biosensors Fundamentals and Applications, chapt. 15 (Turner, et al, eds., Oxford University Press, 1987); Mell, et al, “Amperometric Response Enhancement of the Immobilized Glucose Oxidase Enzyme Electrode,” Analytical Chemistry, Vol. 48, pp. 1597-1601 (September 1976); Mell, et al, “A Model for the Amperometric Enzyme Electrode Obtained Through Digital Simulation and Applied to the Immobilized Glucose Oxidase System,” Analytical Chemistry, Vol. 47, pp. 299-307 (February 1975); Myland, et al, “Membrane-Covered Oxygen Sensors: An Exact Treatment of the Switch-on Transient,” Journal of the Electrochemical Society, Vol. 131, pp. 1815-1823 (August 1984); Bradley, et al, “Kinetic Analysis of Enzyme Electrode Response,” Anal. Chem., Vol. 56, pp. 664-667 (1984); Koichi,“Measurements of Current-Potential Curves, 6, Cottrell Equation and its Analogs. What Can We Know from Chronoamperometry?” Denki Kagaku oyobi Kogyo Butsuri Kagaku, Vol. 54, no.6, pp. 471-5 (1986); Williams, et al, “Electrochemical-Enzymatic Analysis of Blood Glucose and Lactate,” Analytical Chemistry, Vol. 42, no. 1, pp. 118-121 (January 1970); and, Gebhardt, et al, “Electrocatalytic Glucose Sensor,” Siemens Forsch.-u. Entwickl.-Ber. Bd., Vol. 12, pp. 91-95 (1983). Commercial kits for measuring blood glucose are available, e.g., Accu-Chek Active System (Roche Diagnostics), Medisense Optium Blood Glucose Monitor Kit (Abbot Diagnostic Division), or BD Logic Blood Glucose Monitor (Becton, Dickinson). Insulin measurement kits, e.g., the AutoDELFIA Insulin Kit (Perkin Elmer Life Sciences), are also commercially available. Similarly, kits to measure plasma lactate levels, e.g., AccuTrend Lactate (Roche Diagnostics), are readily available. There are a number of instruments for the determination of the concentrations of biologically significant components of bodily fluids, such as, for example, the glucose concentration of blood.

G. Quantifying the Predictive Value of Biomarkers

The inventors have established the correlation between a biomarker's prediction of insulin sensitivity and a simulated GIR for a prevalence weighted virtual patient cohort. A novel methodology based on the commonly used ROC curves (Swets, Science 240:1285-1293 (1988); Hanley, Crit Rev Diagn. Imaging 29:307-335 (1989); Zweig and Campbell, Clin Chem 39:561-577 (1993); Boyd, Scand. J Clin Lab Invest Suppl 227:46-63 (1997)) was developed to quantify the clinical value of the proposed biomarkers.

Typically, ROC analyses focus on a fixed clinical characteristic of pathology and seek optimum values from a clinical test(s) to most reliably distinguish disease from health. A reliable test is one for which the sensitivity is large (i.e., the proportion of healthy people predicted to be healthy), and the specificity is small (i.e., the proportion of unhealthy people predicted to be healthy).

There are not many well established techniques for using ROC curves to evaluate predictions of a continuous variable, such as the biomarkers examined in this project. Bouma and colleagues (Diabetes Care 22:904-7 (1999)) approached the problem by examining ROC curves for several threshold values of a marker predictive of glycosylated hemoglobin (HbA1c). Although they reported correlations between their candidate biomarker (glucose) and HbA1c, and found a best-fit line for describing the relationship, they did not use that information when creating their ROC curves. The analysis presented herein, generalizes the technique to incorporate the fitted information into the ROC analysis. Briefly, the strategy is to select, for any given candidate threshold of insulin sensitivity, a corresponding point on the predictor axis and develop quadrants in the response plane that can be categorized as “True Positives,” “True Negatives,” “False Positives,” and “False Negatives,” and from that structure, generate a sensitivity and specificity value for the ROC.

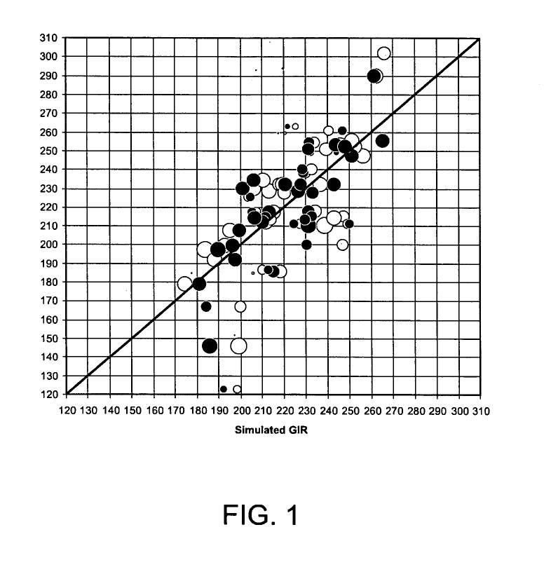

The predictive capacity of QUICKI, HOMA, fasting insulin, and an optimal fasting biomarker were compared by correlating their readouts with the quantification of insulin sensitivity via a hyperinsulinemic-euglycemic clamp. FIG. 1 shows the predictions derived from the biomarker versus the simulated euglycemic clamp pump rate, the GIR, for the optimal fasting biomarker. This marker included fasting measures of insulin, lactate, and glucose. The figure also shows a biomarker based on insulin alone, which has the greatest role in the behavior of this three-variable biomarker. The coefficient of determination, R², for the proposed biomarker is 59%, while for fasting insulin alone it is 45%.

To provide a broader perspective on these results, the results were evaluated in terms of sensitivity and specificity by extending traditional ROC analyses to deal with continuous variable readouts such as insulin sensitivity. This methodology is illustrated here with an example based on two continuous prevalence distributions of predicted and observed outcomes—one representing the case observed in investigating biomarkers for fasting subjects and the other representing a case wherein no correlation can be ascribed to a potential biomarker.

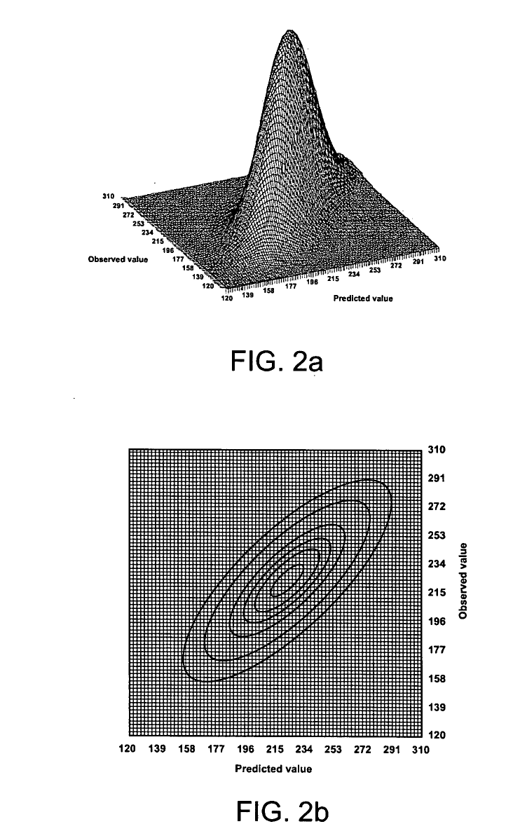

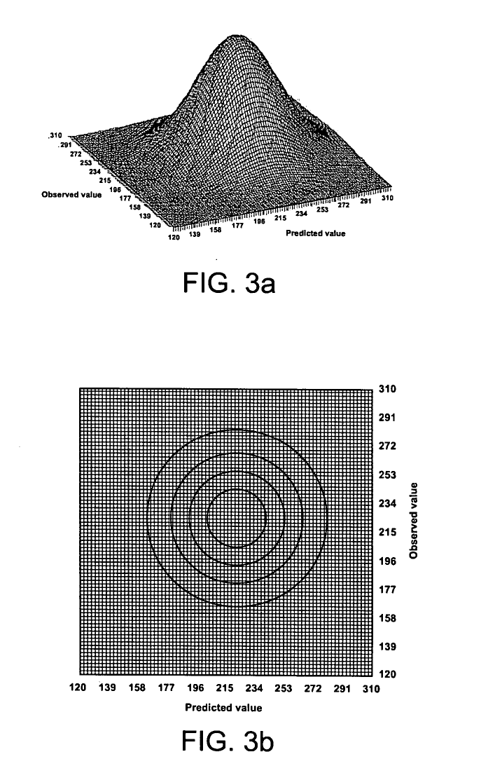

Consider the two idealized biomarkers here. In the first, the predicted and true values have an R² of 59%, similar to that of the optimal fasting biomarker. In the second, the proposed biomarker and the GIR are completely uncorrelated. Assuming that virtual patient prevalences are distributed in bivariate normal distributions, with means and standard deviations as for the simulated populations, one can visualize these relationships graphically. FIG. 2 shows a continuous bivariate Normal prevalence distribution approximating the optimal fasting biomarker, both in a three-dimensional view and as a contour plot, which is more directly comparable to FIG. 1. FIG. 3 also shows a continuous bivariate Normal prevalence distribution for a “biomarker” that has zero correlation as a three-dimensional view and in a contour plot. This zero correlation case is useful for reference, as shown below.

A standard ROC curve would be derived from FIG. 2 by selecting a threshold for the true observed value, and then plotting the following ratios as a function of threshold for the predicted values:

$$Sensitivity = \frac{TP}{TP + FN} \\ 1 - Specificity = \frac{FP}{TP + TN} \qquad Equation 1$$

where TP=True Positive, FP=False Positive, TN=True Negative, and FN=False Negative. As illustrated in FIG. 2, this corresponds to fixing the horizontal line and moving the vertical line, evaluating Equation 1 at each position of the vertical line.

However, when evaluating the performance of a predictor of a continuous quantity, only one value on the ROC curve is appropriate, i.e., the one where the threshold for the predicted value is equal to the true value. In FIG. 2, this corresponds to moving both the vertical and horizontal lines simultaneously, such that they intersect on the diagonal, and evaluating Equation 1 at each position.

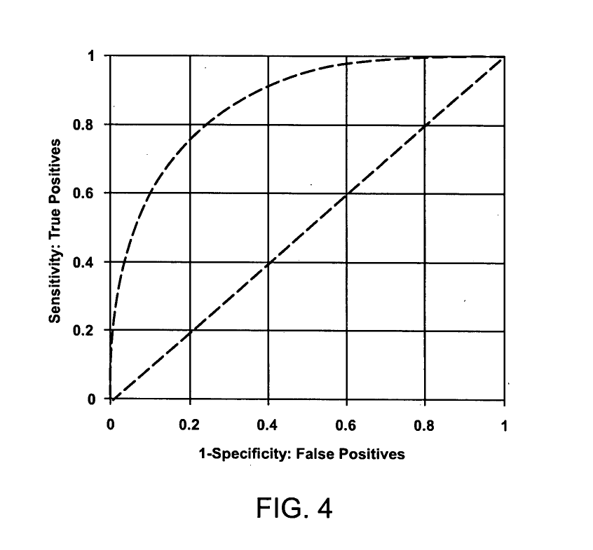

FIG. 4 shows the resulting set of ROC values for the two distributions. As can be seen, these idealized results look similar to a traditional ROC curve. The uncorrelated biomarker simply traces the diagonal, i.e., the rates of true and false positives are the same. Such a biomarker carries no information. The curves show that the best predictions from the high-R² biomarker occur for values inside the bulk of the prevalence distribution. As might be expected, because there are very few patients near the edges of the distribution, the discrete nature of the data at these extremes ensure that the contributions of individual patients is more likely to skew the biomarker's sensitivity and specificity values. The ranges of prevalence where these effects are minimized will be termed the “dynamic range” of the biomarker.

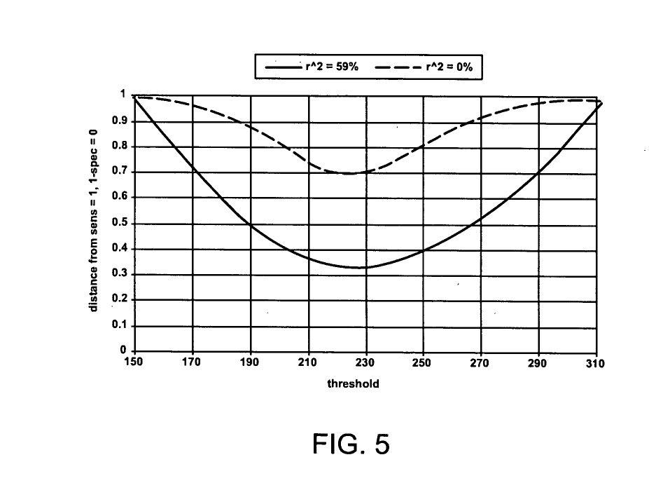

An additional plot proves particularly useful when analyzing virtual patient data. The plot is generated by graphing the distance from the upper left corner of FIG. 4 as a function of candidate threshold level. In typical ROC analyses, any diagnostic that yields a point in the upper left hand corner of the plot yields the best possible case, i.e., all true positives and no false negatives. Thus, the distance from this ideal point can act to quantify the distance from “perfection” for each measurement value. FIG. 5 shows this plot for the two cases shown above. It should be noted that even the uncorrelated biomarker shows some shape on this graph because the distance to the upper right corner varies along the diagonal. The more highly correlated biomarker approaches the upper left corner more quickly and gets closer to it, as indicated by the steeper curve.

### IV. EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### Type 2 Diabetic Virtual Patients

A cohort of ten diabetic virtual patients was chosen to represent the spectrum of phenotypes and pathophysiologies observed in clinical patient populations. The clinical characteristics of these patients were produced by introducing a number of lesions known or suspected to be associated with type 2 diabetes, including various insulin secretion profiles and different combinations of insulin resistance in various tissues. A summary of virtual patient characteristics taken after an overnight fast is shown in Table 1 below.

TABLE 1
General characteristics of type 2 diabetic virtual patients
measured after an overnight fast.

| Virtual | Body Weight | Glucose | | HbA1c | FFA | TG | HGO |
|---|---|---|---|---|---|---|---|
| Patent | (kg) | (mg/dl) | Insulin (μU/ml) | (%) | (μM) | (mg/dl) | (mg/min) |
| #1 | 85 | 130 | 16.5 | 7.5 | 640 | 160 | 163 |
| #2 | 85 | 144 | 21.0 | 8.2 | 641 | 182 | 161 |
| #3 | 70 | 150 | 11.9 | 8.6 | 638 | 185 | 153 |
| #4 | 85 | 155 | 16.4 | 9.1 | 657 | 99 | 154 |
| #5 | 85 | 166 | 10.5 | 9.2 | 865 | 202 | 158 |
| #6 | 85 | 172 | 17.5 | 9.8 | 706 | 135 | 154 |
| #7 | 85 | 181 | 14.8 | 8.6 | 749 | 120 | 158 |
| #8 | 70 | 192 | 18.5 | 9.3 | 713 | 133 | 178 |
| #9 | 85 | 198 | 18.3 | 9.2 | 785 | 157 | 210 |
| #10 | 70 | 206 | 24.0 | 9.2 | 765 | 163 | 192 |

The Metabolism PhysioLab platform was initially developed using a representation of normal physiology. The normal virtual patient was created using parameter sets, each of which mathematically describes a relationship between physiological variables relevant to metabolism. For example, the parameter set for liver glycogenolysis describes the relationships between glycogenolysis rate and plasma glucose, insulin, glucagon, and epinephrine. Each physiological relationship is calibrated using non-proprietary data, with the overall behavior of the normal virtual patient (who is the sum of many parameter sets) validated using experimental protocols that represent complex behavior such as the response to mixed meal feeding.

Type 2 diabetic virtual patients were created by manipulating each parameter set in the normal subject to describe the changes in relationships between physiological variables that occur with diabetes. For example, the dose response curve for the effect of insulin on muscle glucose uptake may be altered to represent reduced insulin sensitivity. Each virtual patient was then validated in a variety of experimental protocols to confirm that its behavior is consistent with reported human clinical data. For example, the diabetic virtual patient may have reduced glucose uptake and elevated hepatic glucose output in a hyperinsulinemic euglycemic clamp when compared to the normal patient, but the magnitude of these changes must be within reported ranges.

The Metabolism PhysioLab platform is completely observable, meaning that every entity represented in the platform can be sampled continuously during the course of an experiment. For example, Entelos scientists are able to measure plasma, portal, hepatic, sinusoidal, and intracellular glucose and insulin concentrations during many different types of experiments. For the purposes of this project, an illustrative sample of measurements of interest was chosen.

A series of in silico experiments were performed to characterize in detail the behavior of each type 2 virtual diabetic patient. The output from these simulations consists of computed values for metabolite concentrations (e.g., plasma glucose concentration) and processes (e.g., rate of muscle glycogen synthesis), taken at time points of clinical interest.

1. Twenty-Four Hour Fast

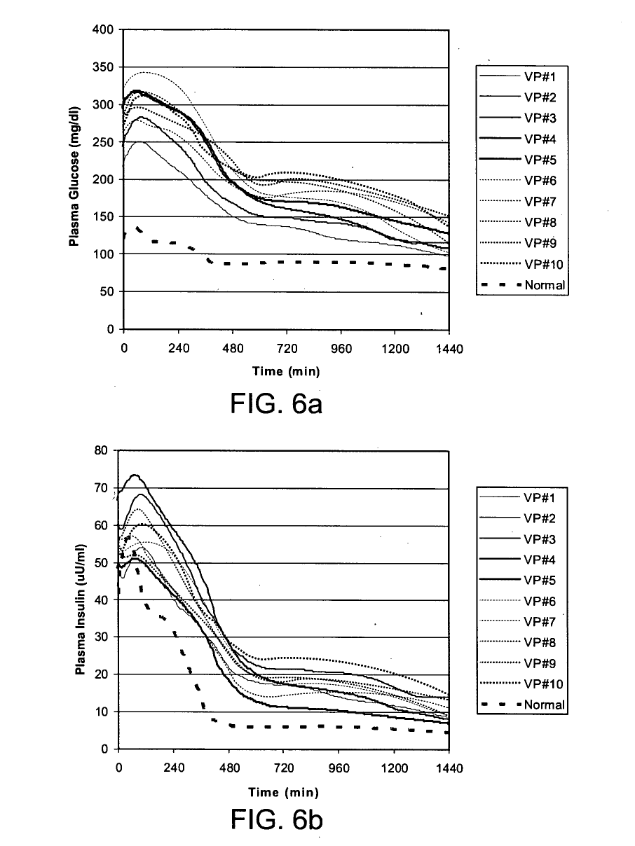

The simulated twenty-four-hour fasting protocol begins at the time of the last meal. In a typical subject with type 2 diabetes, plasma glucose and insulin concentrations decrease over time in response to extended fasting and eventually approach normal levels (Gannon et al., Metabolism 45:492-497 (1996)). FIG. 6 illustrates that glucose and insulin concentrations decreased over time with fasting in the diabetic virtual patients, but did not decrease below normal values. This demonstrated that the dynamic representation of fasting was appropriate in these patients and spanned an appropriate diversity of patient response.

2. Mixed Meal Consumption

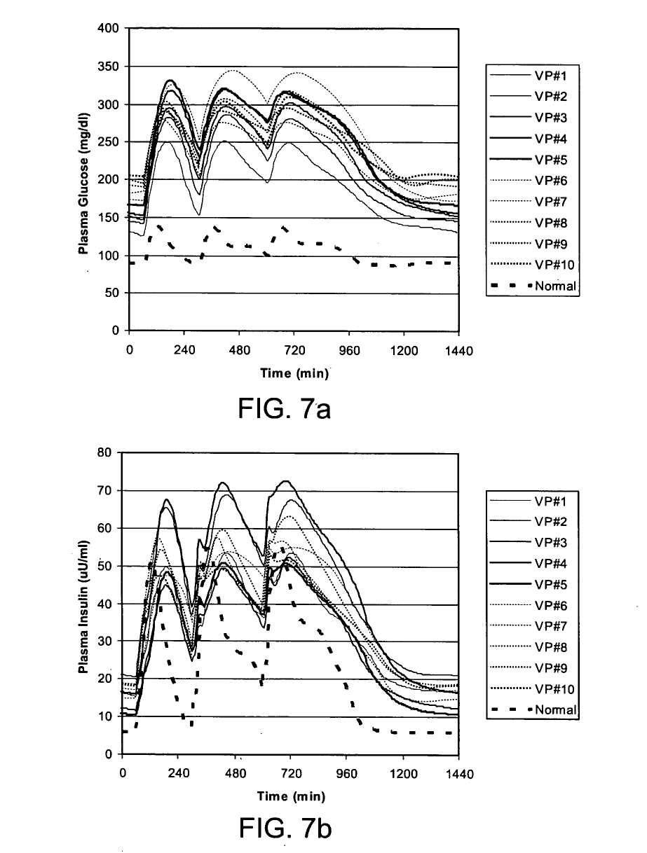

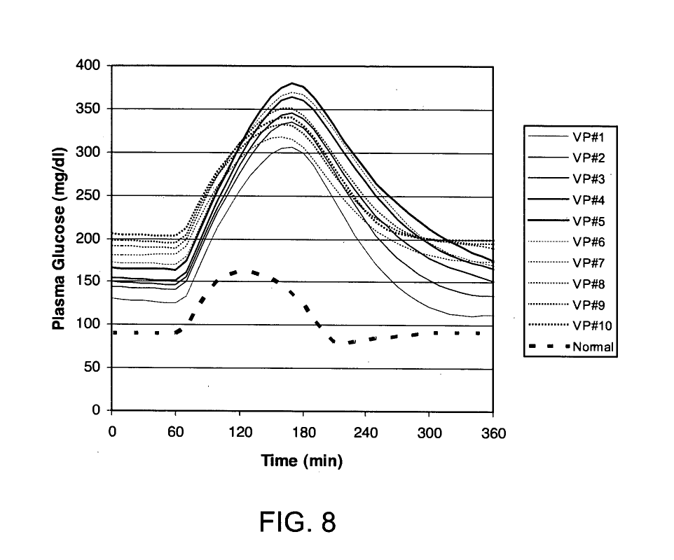

Mixed meal consumption over a twenty-four-hour period represents a complex series of processes that includes gastric emptying and intestinal absorption and the effects of various circulating nutrient and hormonal influences on tissue nutrient uptake. While all the virtual patients had a reasonable response to the mixed meal protocol (Polonsky et al., N. Engl. J Med 318:1231-1239 (1988)), the diversity of that response is illustrated by changes in plasma glucose and insulin (FIG. 7).

3. Oral Glucose Tolerance Test

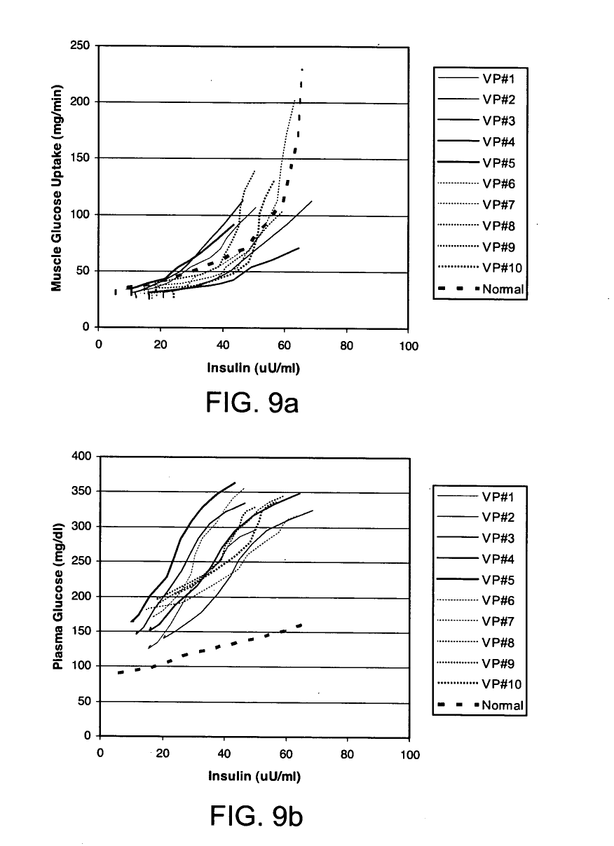

Oral glucose tolerance test (OGTT) is a measure of the ability of the body to dispose of an oral glucose load. An increase in plasma glucose concentration above initial levels (FIG. 9) indicates a reduction in glucose tolerance, which is characteristic of type 2 diabetes (Fery et al., Metabolism 42:522-530 (1993)). Under these conditions, much of the glucose is disposed of by skeletal muscle. A rightward shift in the dose response curve of muscle glucose uptake versus insulin is an indication of reduced insulin sensitivity (i.e., insulin resistance).

FIG. 9 illustrates that all of the virtual patients chosen for this project had various degrees of insulin resistance during an OGTT. It should be noted however that the diabetic patients had higher than normal plasma glucose concentrations at any given insulin concentration, and that plasma glucose increases its own disposal. Under these conditions, conclusions about insulin sensitivity are difficult to draw. Therefore, determinations of insulin sensitivity should be made under conditions in which both plasma glucose and insulin are controlled, such as during a hyperinsulinemic euglycemic clamp.

4. Intravenous Glucose Tolerance Test

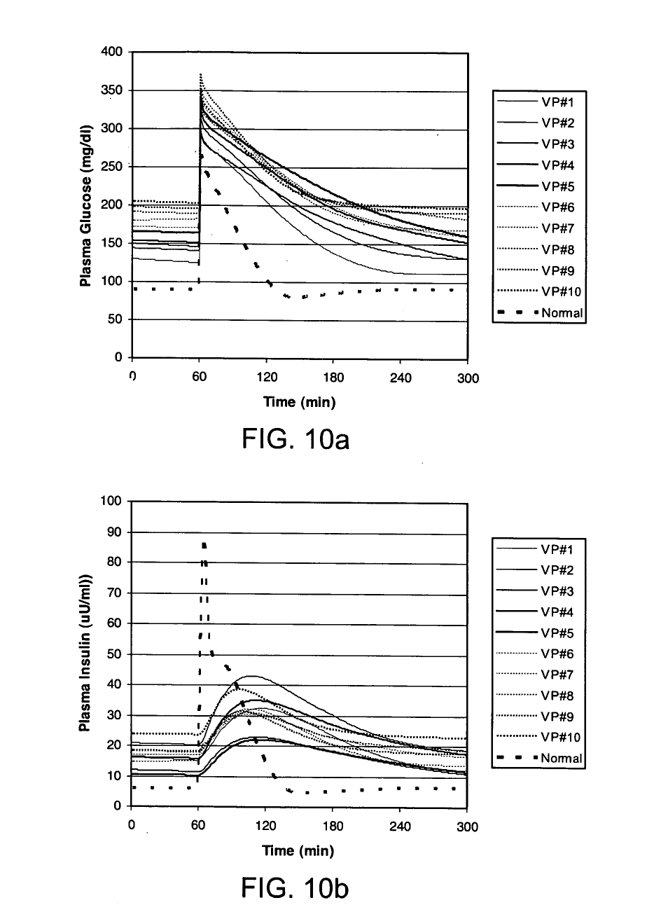

An Intravenous glucose tolerance test (IVGTT), like the OGTT, is a measure of the ability of the body to dispose of a glucose load. In contrast to the OGTT, the IVGTT avoids the influence of gastrointestinal factors such as glucose absorption and incretin release. In addition, the rapid rise in plasma glucose (FIG. 5) induced by intravenous injection of glucose allows the examination of first-phase insulin release, which is dysregulated very early in the pathogenesis of type 2 diabetes (Kahn et al., J Clin Endocrinol Metab 86:5824-5829 (2001)). Of the ten type 2 diabetic virtual patients tested, none had appreciable first-phase insulin release, while second-phase insulin release was variable between the patients (FIG. 10).

5. Hyperinsulinemic Euglycemic Clamp

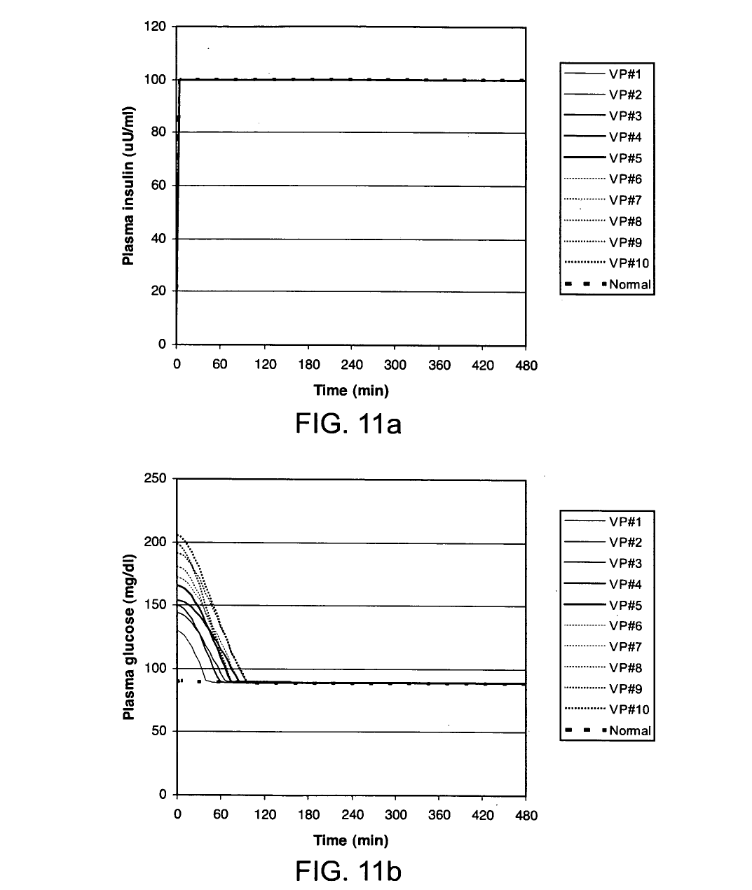

The hyperinsulinemic euglycemic clamp is considered the best test of insulin sensitivity (Defronzo et al., J Clin Invest 76:149-155 (1985)). In this method, a constant insulin infusion in overnight fasting subjects produces a state of hyperinsulinemia (˜100 uU/ml) sufficient to reduce plasma glucose concentration (FIG. 6). A glucose infusion is then initiated and adjusted to maintain plasma glucose concentration in a state of euglycemia (90 mg/dl in our protocol, FIG. 11).

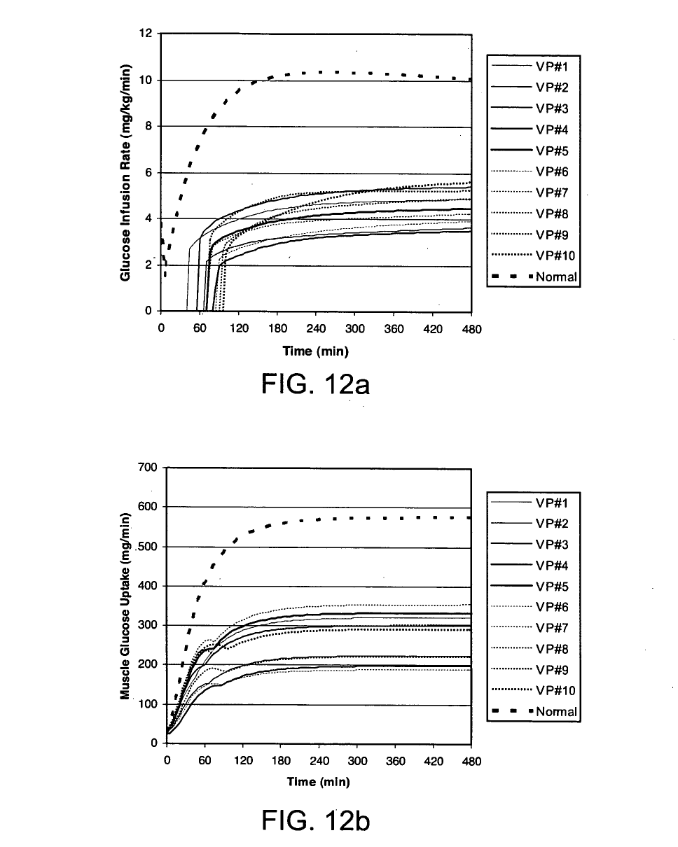

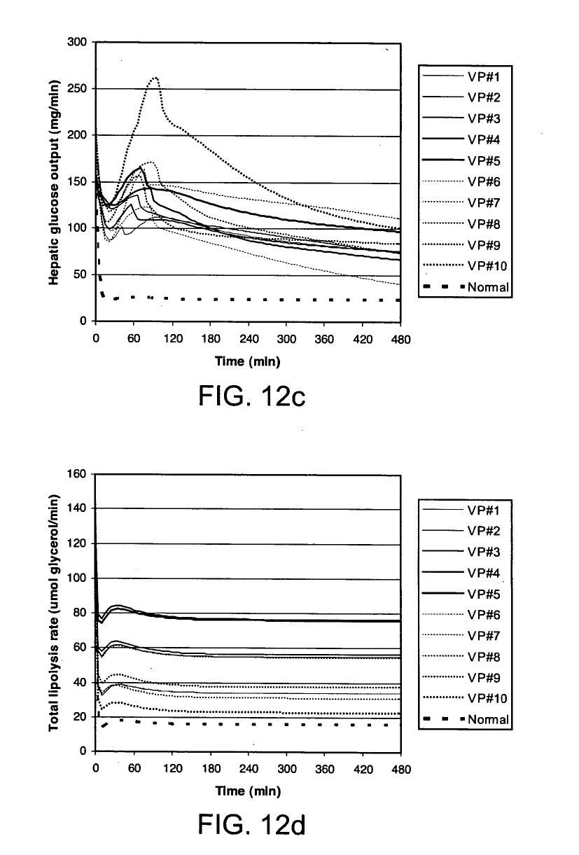

The hyperinsulinemic euglycemic clamp protocol is designed so that insulin sensitive processes are measured and compared between subjects at equivalent plasma insulin and glucose concentrations. The rate of glucose infusion required to maintain euglycemia is a measure of insulin sensitivity. The higher the glucose infusion rate required to maintain euglycemia the greater the glucose disposal and suppression of endogenous glucose production. Measurements of muscle glucose uptake, hepatic glucose production, and adipose tissue lipolysis under these conditions are indicators of tissue specific insulin sensitivity. Subjects with type 2 diabetes typically display insulin resistance for each these processes, although the nature and degree of resistance among the various tissues varies considerably between subjects. This phenomenon was demonstrated by the responses in the virtual patients (FIG. 12).

6. Hyperglycemic Clamp

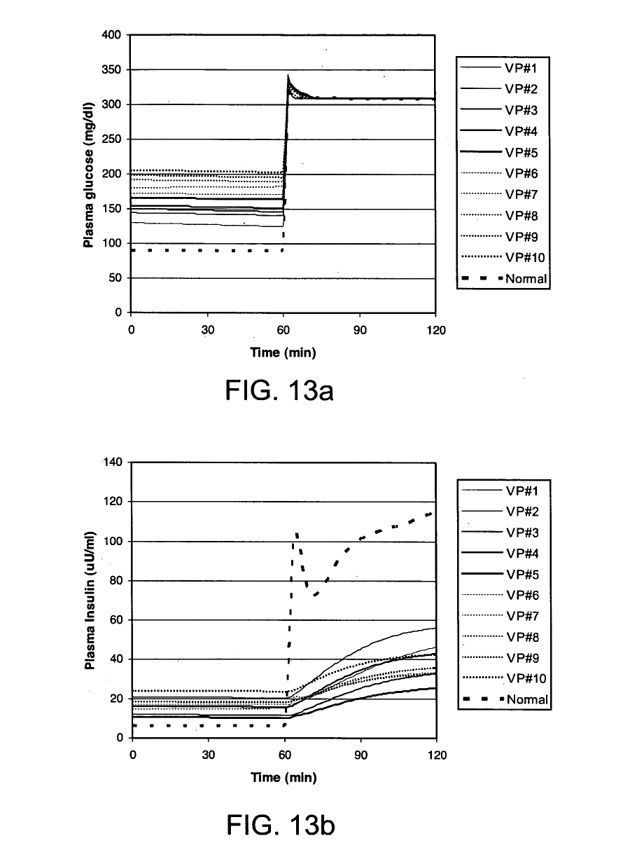

The hyperglycemic clamp is primarily a measure of insulin secretion. Like the IVGTT, the hyperglycemic clamp uses an intravenous infusion of glucose and can thus be used to demonstrate first-phase insulin secretion (Van Haeften et al., Eur J Clin Invest 21:168-174 (1991)). In contrast to the IVGTT, the hyperglycemic clamp provides equal glucose concentrations between experimental subjects (FIG. 13) and thus a more controlled comparison of insulin secretion rates. The increment in plasma insulin concentration over basal concentration in the first ten minutes of the clamp is considered a measure of first-phase insulin secretion. This response disappears early in the pathogenesis of type 2 diabetes and was largely absent in the virtual patients (FIG. 13). Second-phase insulin secretion is defined as the increment in plasma insulin concentration from ten to sixty minutes after the start of glucose infusion. The virtual patients displayed a range of second-phase insulin secretion that is reflective of patient diversity.

### B. Example 2

### Virtual Patient Selection

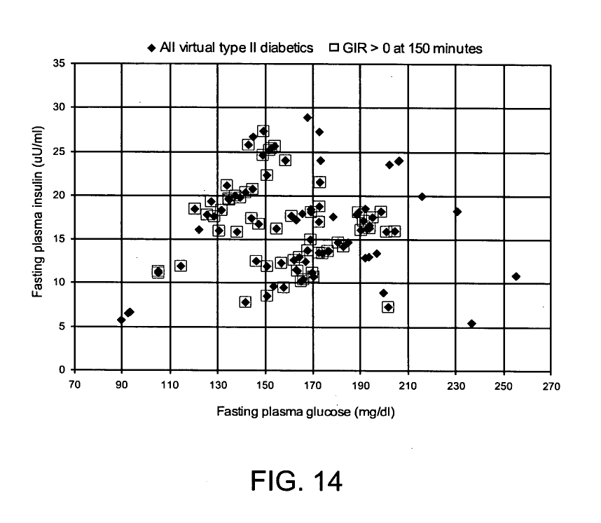

An overview of the virtual patients available for analysis is shown in FIG. 14 and Table 2. FIG. 14 shows fasting insulin and glucose values for each patient, as well as the values for the subpopulation used in the analysis. Table 2 shows the distribution of severities of diabetes and weight characteristics. In parentheses are the corresponding numbers for virtual patients from each class used in this study.

TABLE 2
Number of virtual patients with indicated weight and type 2 severities

| | | | |
|---|---|---|---|
| Weight (Kg) | 70 | 85 | 100 |
| % body fat | 20 | 30 | 40 |
| BMI (kg/m²) | 24 | 30 | 35 |
| severe | | 2 (0) | 1 (0) |
| moderate | 8 (6) | 59 (38) | 5 (3) |
| mild | | 11 (10) | 1 (1) |
| pre-diabetic | | 4 (4) | |
| non-diabetic | 1 (0) | 1 (0) | 1 (0) |

Euglycemic clamp simulations were analyzed for all virtual patients at 60, 80, and 100 μU/ml insulin. The higher and lower values were included in this study as proxies for variations in insulin clearance. The GIR (taken as the average infusion rate over the 150 to 180 minute interval corrected for body mass) was significantly higher for the non-diabetics than the diabetic populations, so these non-diabetics were excluded from the patient pool. In addition, approximately one third of the virtual patients did not reach euglycemia in 150 minutes at 60 μU/ml insulin, five of these did not reach euglycemia at 80 μU/ml, and three of these latter patients did not reach it at 100 μU/ml. Each of these patients also was excluded from subsequent analyses. Typically, glucose pump start times correlated rather strongly with fasting glucose levels. Most of the excluded patients are those with high fasting glucose compared to others at similar insulin levels (FIG. 14).

The virtual patient pool that exhibited glucose pump activity before 150 minutes at 60 μU/ml insulin made up the analysis set for this study. Infusion rates of 60 μU/ml have been used in certain protocols (Bonora et al., Diabetic Med 19:535-542 (2002); Mitrakou et al., J Clin Endocrin Metab 75:379-382 (1992)), but this rate is lower than that typically reported for hyperinsulinemic-euglycemic clamps. This is the first human clinical constraint applied to the virtual patient pool for this study. It provided a uniform pool of patients that allows examination of the effects of insulin pump rates (effectively a way of varying insulin clearance rates) on observed correlations.

### C. Example 3

### Analysis of Previously Studied Biomarkers

Much work has been done on finding measurements to predict insulin sensitivity. Wallace and Matthews (2002) and Radziuk (2000) provide useful reviews, and the series of letters in response to Matsuda and DeFronzo (1999) illustrates some of the current debate.

Much of the discussion of insulin sensitivity biomarkers focuses on HOMA, which is simply proportional to the product of fasting insulin and glucose, and QUICKI, which is essentially the reciprocal of the log of HOMA (Matthews et al., 1985; Katz et al., 2000):

$$\begin{matrix}HOMA = \frac{[ fasting insulin ( uU \text{/} ml ) ] \times [ fasting glucose ( mg \text{/} dl ) ]}{405} \\ 34\end{matrix}$$

$$QUICKI = \frac{1}{\begin{matrix}log ( [ fasting insulin ( uU \text{/} ml ) ] \times \\ [ fasting glucose ( mg \text{/} dl ) ] )\end{matrix}}$$

A first test of the clinical relevance of the virtual patient pool was a comparison to clinical reports of correlations between HOMA or QUICKI and hyperinsulinemic-euglycemic or hyperinsulinemic-isoglycemic clamp results.

Two recently published comparisons of HOMA and hyperinsulinemic-euglycemic clamp measurements (Bonora et al., 2000; Rabasa-Lhoret et al., 2003), and one study of QUICKI (Katz et al., 2000) emphasized correlations between the log of HOMA and insulin sensitivity. This is appropriate, since Bonora et al. (2000) showed a hyperbolic relationship between HOMA and glucose disposal rates.

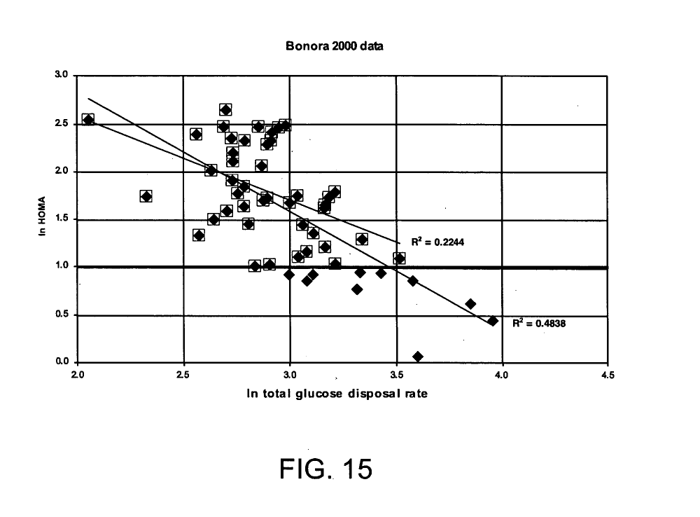

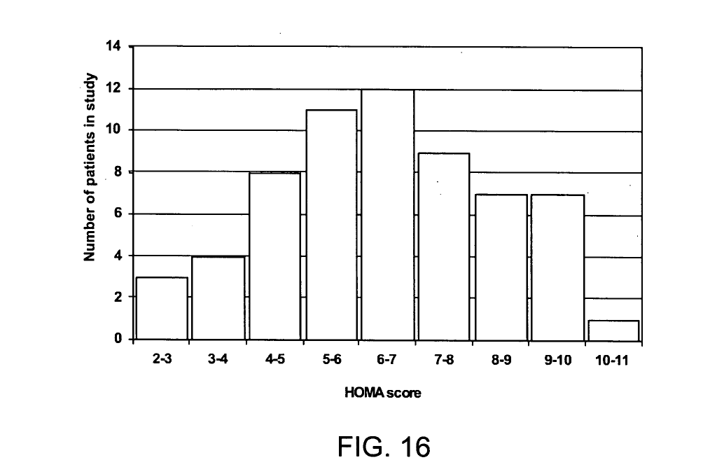

Bonora et al. (2000) executed a hyperinsulinemic clamp protocol on type 2 diabetics and non-diabetics with insulin infusion rates of 20 mU/min/m² body area, which corresponds approximately to 60 μU/ml of insulin in our virtual patients. They used tracers to measure glucose clearance. FIG. 15 shows their data, for which they report an R² of 48% for the entire diabetic population. The HOMA for the average diabetic in their study is 6.9. As shown in FIG. 16, the distribution of virtual patient HOMA scores is consistent with this average. However, Bonora et al. (2000) included several subjects with HOMA scores considerably below those of the virtual patients, which contribute strongly to the R². Considering only the subset of subjects with HOMA scores comparable to those of the virtual patient population, the R² drops to 22%.

Rabasa-Lhoret et al. (2003) reported an R², 56%, for the correlation between log HOMA and GIR similar to that observed in Bonora et al. (2000). In their study, they performed a hyperinsulinemic-euglycemic clamp on type 2 diabetics with a considerably higher insulin pump rate of 75 mU/min/m² body area, which corresponds approximately to 165 μU/ml of insulin in the virtual patient cohort. They also reported an identical correlation with QUICKI. They did not provide individual data for the diabetics, so it is difficult to compare in detail this result with the target virtual patients.

### D. Example 4

### Prevalence Weighting

Katz et al. (2000) performed hyperinsulinemic-isoglycemic clamps at insulin infusion rates of 120 mU/min/m² body area and reported an R² of 49% between QUICKI and glucose pump rates in diabetics. The R² for the virtual patients under the same protocol was <1%. Even when the analysis was restricted to the subset of subjects with QUICKI scores similar to those observed in the virtual patient cohort, the R² only improved to 33%. These clinical data suggest that the virtual patient population, aimed at representing diversity of underlying pathophysiologies, required normalization to more adequately represent the underlying prevalence of observed phenotypes in the actual clinical population. To do this, a novel methodology, based on the same statistical assumptions of normality and proportionality that underlie the methods and techniques of Analysis of Covariance was developed. From this method, an estimated probability of observance for each virtual patient was calculated.

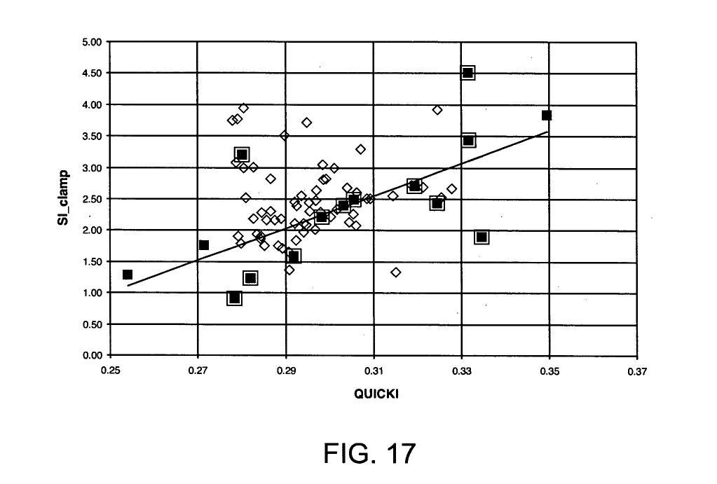

FIG. 17 shows the data that was used to make these calculations (Katz et al., 2000). The figure plots the data taken in type 2 diabetics and the corresponding virtual patients. These data were not used to develop the virtual patients. Therefore, the data shown in FIG. 17 provide additional validation of the virtual patients representing actual subjects. The degraded correlation within the unweighted virtual patient population appears to be mainly caused by the inclusion of too many subjects with low QUICKI and high glucose infusion rates.

Rather than simply eliminate these virtual patients, and thus bias the results, relatively simple, objective approach was used to assign prevalence weights to the virtual patients. It was assumed that the prevalence of the virtual patients is distributed normally about a least-squares line through the population, with a constant standard deviation for the distribution (i.e., the assumption of homoschedasticity). Thus, one was able to simultaneously infer the weighted least squares fit to the data and the appropriate weightings simultaneously. These minimal constraints gave an R², slope, and intercept consistent with the clinical data and thus stabilized the resulting parameter estimation problem.

The first constraint applied to the weighting is a penalty for deviation from uniformity. To get convergence, this penalty was approximately equal to the sum of squared errors. The resulting weighted R² is 48%. However, the slope of the line was not within the 99% confidence interval of the line through Katz et al.'s data.

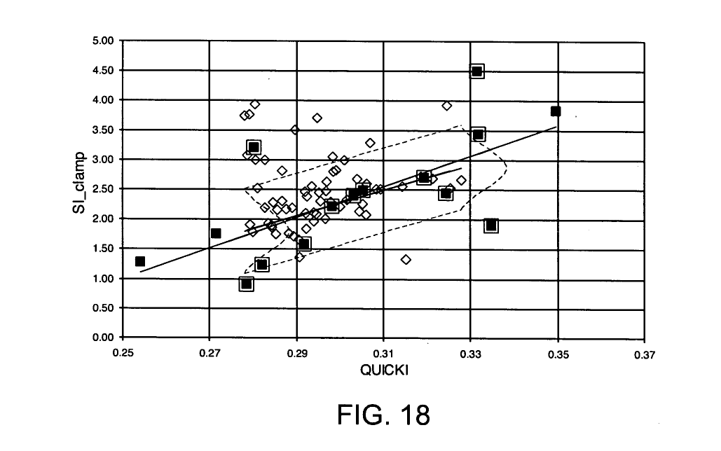

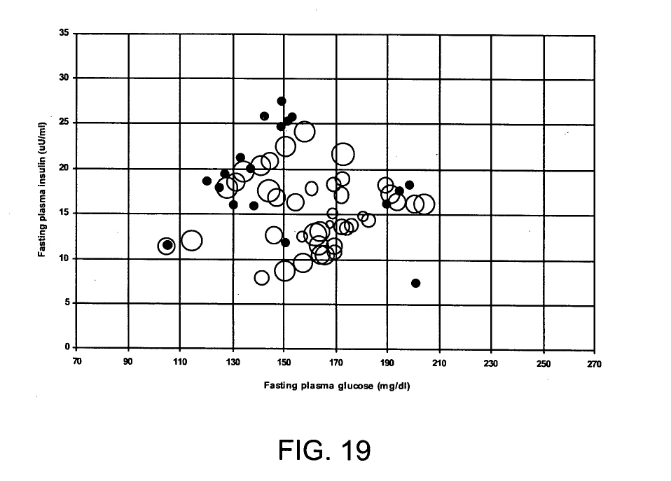

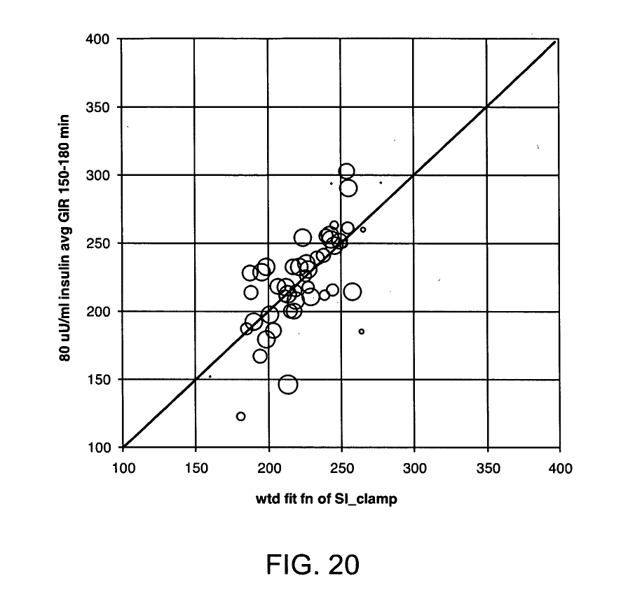

Further study showed that if one simultaneously applies penalties to deviations from uniform weighting and deviation from the R² of Katz et al's data, one can derive lines consistent with their R², slope, and intercept. FIG. 18 illustrates the preferred weighting scheme, with an R² of 48% and a slope and intercept comparable to (i.e., within the 90% confidence limits) the line through Katz et al.'s data.

FIG. 11 shows the distribution of weightings among the patients, along with their fasting glucose and insulin values. To explore the sensitivities of the estimated correlations to the weighting scheme, additional correlations were calculated where the width of the normal distribution shown in FIG. 18 was expanded by 1.5 and 2×. Another study was performed with a weighting scheme derived from an initial fit having a 33% R² and a slope and intercept within the (larger) 99% confidence interval of the line through Katz et al.'s data. Briefly, the R² degraded from 59% to 42% with increasing width of the standard deviation and was reduced to 49% when one employs the secondary linear fit as a starting value vector.

### E. Example 5

### Pairwise Correlation Analysis

The biomarker development effort next examined the physiological measures shown in Table 3 after an overnight fast. Measures that varied by less than 5% (e.g., norepinepherine) across the virtual patient population were eliminated as unlikely to be practically measured. Prevalence-weighted correlations between the remaining quantities and GIR were computed and ranked (Table 4).

TABLE 3
Overnight fasting measures investigated during this biomarker
development effort

| | | |
|---|---|---|
| amino acids (total) | glucose | % body fat |
| C-peptide | glycerol | body mass index |
| epinephrine | HBA1c | body surface area |
| free fatty acids | Insulin | body weight |
| fructosamine | lactate | lean body mass |
| GIP (inactive) | norepinepherine | |
| GIP: total | TG: chylomicron | HOMA |
| GLP-1 (active) | TG: total | QUICKI |
| GLP-1 (inactive) | TG: VLDL | revised QUICKI |
| GLP-1: total | glucose deviation from avg | |
| | glucose | |

Table 4 shows the physiological quantities examined by multilinear regression analysis, along with their bivariate R² values. Certain quantities were excluded from further analysis (e.g., C-peptide, fructosamine) if they correlated strongly with those already identified as predictive. Table 4 also shows, for comparison, correlations with QUICKI and values from a regression without prevalence weighting. The remaining quantities either showed too little variation or insignificant correlation with GIR.

TABLE 4
Significant pairwise correlations between indicated hyperinsulinemic-
euglycemic-clamp GIR values and fasting values of physiological
measures. QUICKI correlation is included for comparison.

| | | Insulin clamp value: | | | | | |
|---|---|---|---|---|---|---|---|
| | | 80 μU/ml | | 60 μU/ml | | 100 μU/ml | |
| | | Data transform: | | | | | |
| | | linear | log | linear | log | linear | log |
| Prevalence | insulin | 45% | 42% | 38% | 32% | 43% | 43% |
| Weighted | FFA | 24% | 25% | 13% | 14% | 26% | 27% |
| | TG (mg/dl) | 16% | 16% | 10% | 10% | 14% | 15% |
| | lactate | 15% | 13% | 17% | 13% | 9% | 8% |
| | HBA1c | 12% | 12% | 10% | 11% | 10% | 10% |
| | glucose | 3% | 5% | 0% | 1% | 7% | 7% |
| | QUICKI | 26% | 22% | 30% | 22% | 20% | 19% |
| Un-weighted | lactate | 19% | 17% | 17% | 13% | 17% | 15% |
| | insulin | 15% | 15% | 19% | 15% | 6% | 7% |
| | TG (mg/dl) | 10% | 12% | 10% | 11% | 8% | 10% |
| | FFA | 4% | 6% | 3% | 6% | 4% | 5% |
| | HBA1c | 0% | 0% | 1% | 3% | 1% | 0% |
| | glucose | 0% | 0% | 1% | 0% | 0% | 0% |
| | QUICKI | 17% | 14% | 24% | 16% | 7% | 6% |

Table 4 yields three important conclusions: First, it shows that the results are relatively insensitive to data transformation. Therefore, the original, untransformed data were used to examine the resulting linear correlations. Second, the correlations were relatively insensitive to changes in the insulin clamp level. Finally, these results show that fasting plasma insulin by itself is a good predictor for GIR.

The prevalence-weighted correlation of the euglycemic clamp data with QUICKI is notably lower than the correlation of isoglycemic clamp data used to determine the prevalence weighting (FIG. 10, Table 4). This is consistent with the discussion of Bonora et al. (2000) above, where it was noted that the clinical data do not support a strong correlation between log HOMA and euglycemic clamp results. The mathematical similarity between log HOMA and QUICKI indicates that the correlation with QUICKI should, likewise, not be strong. The difference in correlation between isoglycemic and euglycemic clamp measurements is caused by the imperfect correlation between the simulated euglycemic and isoglycemic measurements (R²=45%) as shown in FIG. 12. In addition to the different experimental protocols of these measurements, the imperfect correlation is driven by the different definitions of insulin sensitivity: recall that Katz et al. normalized the observed GIR by body weight, individual fasting glucose level held during the clamp, and the change in insulin level during the clamp.

The prevalence weighted correlation of the euglycemic clamp data with QUICKI in Table 4 also is notably lower than the correlation with plasma insulin. This appears to be driven by the fact that QUICKI includes variations in glucose, which are poorly correlated with GIR, in a way that cannot account for the relative importance or independent effects of fasting insulin and glucose. The stepwise regression analysis below shows that glucose levels (HbA1c more specifically) positively correlate with insulin sensitivity.

### F. Example 6

### Multilinear Regression Analysis

Table 5 shows the results of the stepwise regression analysis. The columns of the table show the coefficients of the best fitting lines when one, two, three, or all variables were used in a multilinear regression. The rows correspond to the different variables used in the regressions. The resulting R² is shown for each regression.

TABLE 5
Coefficients and R² values for step-wise regression analysis with preferred
prevalence weightings

| | | | | | | avg glucose - | | | |
|---|---|---|---|---|---|---|---|---|---|
| | Insulin | | | Lactate | | glucose | | Constant | |
| | (μU/ml) | FFA (mg/dl) | TG (mg/dl) | mg/dl) | HBA1c (%) | (mg/dl) (mg/dl) | glucose (mg/dl) | (mg/min) | R² |
| Insulin | −5.67 | | | | | | | 311.3 | 45% |
| Single-variable | −5.67 | | | | | | | 311 | 45% |
| correlations | | 6.36 | | | | | | 95.3 | 24% |
| | | | 0.314 | | | | | 175 | 16% |
| | | | | 16.3 | | | | 80 | 15% |
| | | | | | 12.5 | | | 116 | 12% |
| | | | | | | 0.652 | | 178 | 14% |
| | | | | | | | 0.278 | 179 | 3% |
| Two-variable | −5.52 | 0.419 | | | | | | 301 | 45% |
| correlations | −5.13 | | 0.130 | | | | | 283 | 47% |
| | −5.20 | | | 10.9 | | | | 209 | 52% |
| | −5.34 | | | | 8.68 | | | 232 | 51% |
| | −5.21 | | | | | 0.377 | | 278 | 49% |
| | −5.64 | | | | | | 0.256 | 270 | 48% |
| Three- | −4.74 | | | 12.5 | 10.2 | | | 100 | 59% |
| variable | −4.78 | | | 10.5 | | 0.358 | | 181 | 55% |
| correlations | −5.05 | | | 13.3 | | | 0.370 | 126 | 57% |
| All | −4.72 | 0.561 | −0.0437 | 12.9 | 8.15 | 0.0472 | 0.0776 | 93.2 | 59% |
| variables | | | | | | | | | |

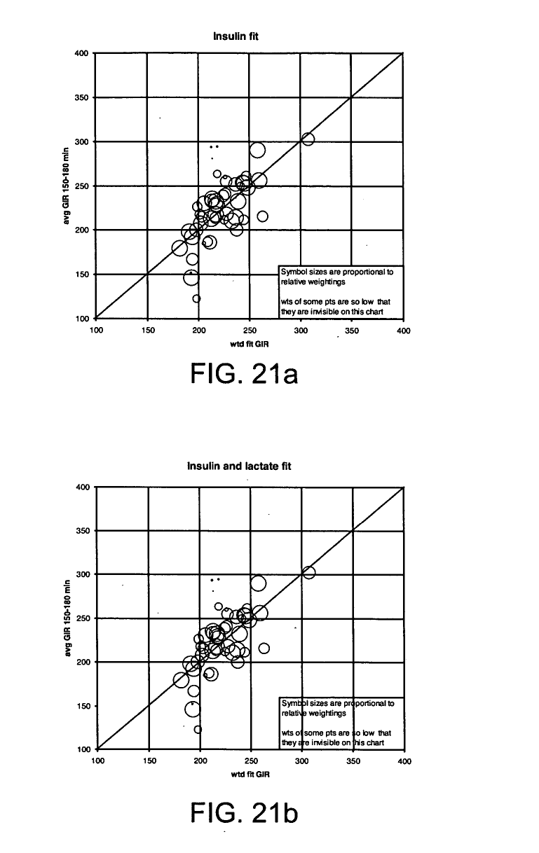

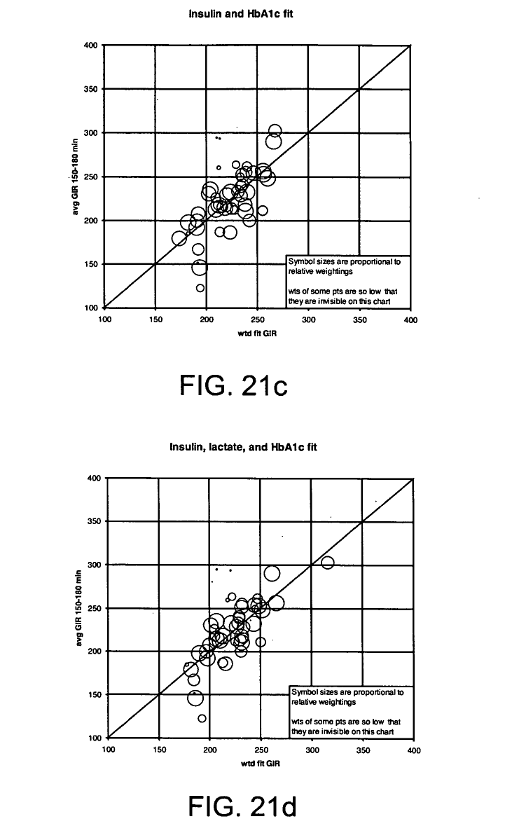

Simulation results show that fasting plasma insulin is by itself quite a good predictor of insulin sensitivity (R²=45%, FIG. 21a). The other plasma quantities in the single-variable-correlations section of the table did not predict insulin sensitivity as well as fasting plasma insulin alone.

To see if the correlation could be improved by a multivariate linear fit, insulin was combined with each of the other variables in a two-variable regression. Significance in this study was not determined by a rigorous, stepwise statistical strategy of model creation (e.g., an F-to enter statistic similar to the discriminant function analysis found in SAS). Rather, the best possible correlation was determined by using all of the strong correlates (R²=59%, bottom row of Table 5). Then the minimal set of quantities that best approached this presumed optimum was considered for further study. Since the strategy is to prioritize efforts for developing improved biomarkers, future research efforts should include a consideration of whether the incremental costs of adding tests would justify, in the clinic, the benefits of improved prediction of insulin sensitivity.

When combined with insulin, both lactate and HbA1c made the biggest improvements in the R² of the regression, adding 6-7% each (FIGS. 21a, 21b). However, their individual incremental effects do not seem to have contributed much to improving the correlation, based on a comparison of FIGS. 21A, 21B, and 21C. Interestingly, both lactate and HbA1c correlated positively with GIR, i.e., increase in either corresponded to increased insulin sensitivity. As is apparent from FIG. 21, this effect is rather small.

Despite their stronger individual correlations with GIR, the fat measures (triglycerides and FFA) did not add to the predictability of insulin in the multivariate analysis. Lactate and HbA1c, in contrast, included notable independent correlations.

Three plasma quantities gave the best R², insulin, lactate, and HbA1c (R²=59%; FIG. 21d). The equation for the regression is
GIR=100−4.74I+12.5L+10.2HbA1c

Fasting plasma glucose appeared to be a reasonable substitute for HbA1c (R²=57%), and might be preferred for practical reasons. Although this biomarker is not dramatically different from insulin alone, it appears to have more discriminatory power, as can be seen by the somewhat more even spread of points along the line of identity in FIG. 21D compared to FIG. 21A. The equation for this biomarker is
GIR=126−5.05I+13.3L+0.370G

### G. Example 7

### Sensitivity of Biomarker to Prevalence Weighting Assumptions

In this project, the underlying assumptions of most interest are those used to define the prevalence weighting scheme (Table 6). The robustness of these results to those assumptions was investigated by examining the effects of scaling by 1.5× and 2×the width of the normal distribution around the line through the virtual patient data. The readouts for this analysis were the coefficients of the three-variable biomarker. The effect of a weighting derived by assuming the R² of the subpopulation in Katz et al.'s data most similar to the virtual patients (33%) was compared to Katz et al.'s whole-population value of 49%. This had the effect of increasing the width of the prevalence weighting 1.5×. Only a doubling of the width of the distribution seemed to have a significant impact on the results.

TABLE 6
Changes in regression coefficients and R² for various
prevalence weightings.

| | Insulin | Lactate | HBA1c | Constant | |
|---|---|---|---|---|---|
| | (μU/ml) | (mg/dl) | (%) | (mg/min) | R² |
| Preferred wtg | −4.74 | 12.5 | 10.2 | 100 | 59% |
| R² = 33%, 1.5x sigma | −4.43 | 13.1 | 9.04 | 103 | 49% |
| 2x sigma of preferred | −4.28 | 15.8 | 5.60 | 107 | 42% |
| wtg | | | | | |

These results show that the conclusions are not strongly dependent on the prevalence weighting scheme. Additionally, the results are relatively insensitive to an approximation used in the simulation of the isoglycemic clamp, which yielded an insulin level ˜5% less than that observed (˜210 μU/ml vs. ˜220 μU/ml).

### H. Example 8

### Biomarkers for Subpopulations

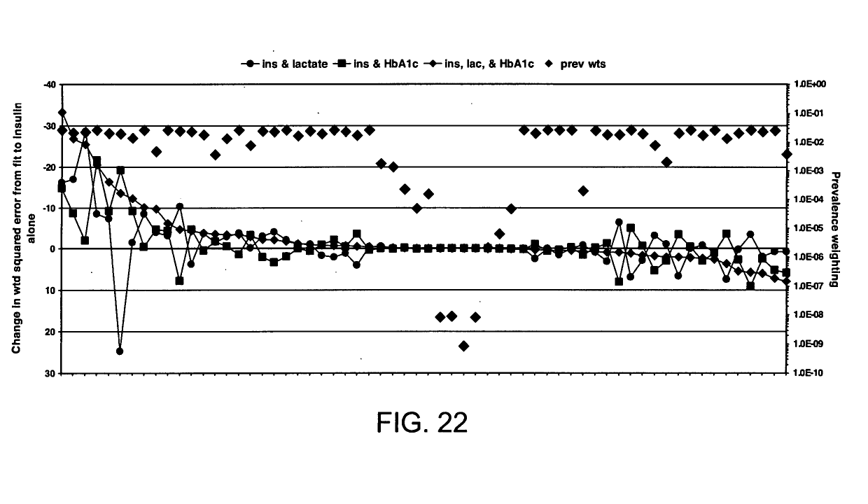

A patient-by-patient analysis of the effects of the steps in the regression on their deviations from the final best fit indicates that there are two virtual-patient subpopulations: those for which HbA1c reduced the error and those for which lactate did. FIG. 22 shows the individual changes relative to insulin alone of the weighted squared deviations from the fitted lines:
wᵢ(y′ᵢ_{_{—}}_{ins+lactate}−yᵢ)²−wᵢ(y′ᵢ_{_{—}}ᵢₙₛ−yᵢ)²
wᵢ(y′ᵢ_{_{—}}_{ins+HbA1c}−yᵢ)²wᵢ(y′ᵢ_{_{—}}ᵢₙₛ−yᵢ)²
wᵢ(y′ᵢ_{_{—}}_{ins+lactate+HbA1c}−yᵢ)²−wᵢ(y′ᵢ_{_{—}}ᵢₙₛ−yᵢ)²
FIG. 14 also shows the individual patient prevalence weightings.

Results from this analysis show a clear negative correlation between the effects of adding HbA1c to the regression and adding lactate. Thus, patients for whom lactate improved the fit had less improvement when HbA1c was added, and vice versa. In many cases, if one improved the fit the other worsened it. For some patients with very little weighting, either lactate or glucose caused no improvement to the regression. The six patients with the lowest weightings differed by less than 1% in their weighted changes to their residuals. These patients were not used for the following analysis because their low weights prevented their having any impact in any case.

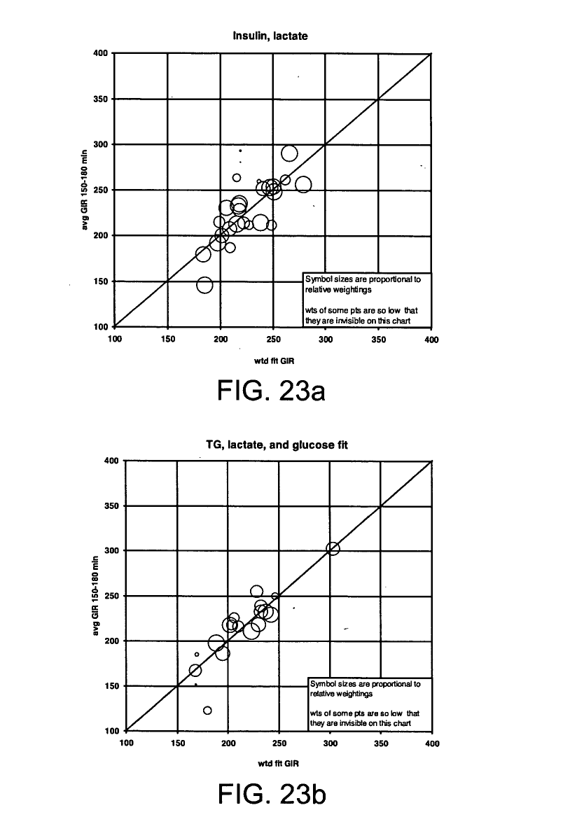

The subpopulation of 32 virtual patients for which lactate improved their fit—and thus presumably had a common physiological mechanism for insulin resistance that involves lactate—were not atypical in their correlation with plasma insulin alone (R²=53%). However, a biomarker profile consisting of a linear combination of insulin and lactate had an impressive correlation within this subpopulation: R²=62% (FIG. 23, Table 7). The equation for the two-variable regression is:
GIR=114.0+23.4L−5.88I

where L represents plasma lactate concentration and I represents plasma insulin concentration. For this subpopulation, including all the quantities from Table 5 increased the R² to 64%.

TABLE 7
Coefficients and R² values for step-wise regression analysis with preferred prevalence
weightings using “lactate-associated” subjects.

| | Insulin | | | Lactate | | avg glucose - glucose | | Constant | |
|---|---|---|---|---|---|---|---|---|---|
| | (μU/ml) | FFA (mg/dl) | TG (mg/dl) | (mg/dl) | HBA1c (%) | (mg/dl) (mg/dl) | glucose (mg/dl) | (mg/min) | R² |
| Single-variable | −5.55 | | | | | | | 312.7 | 53% |
| correlations | | 6.18 | | | | | | 102.5 | 22% |
| | | | 0.15 | | | | | 202.5 | 4% |
| | | | | 14.4 | | | | 100.1 | 3% |
| | | | | | 11.55 | | | 126.4 | 10% |
| | | | | | | 0.614 | | 182.3 | 13% |
| | | | | | | | 0.185 | 196.4 | 2% |
| Two- | −5.88 | | | 23.4 | | | | 114.01 | 62% |
| All | −5.36 | 1.84 | −0.11 | 22.8 | −4.49 | 0.298 | 0.241 | 70.1 | 64% |

The subpopulation of twenty-four patients for which HbA1c improved the fit better than lactate (FIG. 22) also had a similar (slightly higher) correlation between insulin and GIR as the whole virtual patient population (R²=47%). For this subpopulation of patients, there were several potential biomarkers made up of a linear combination of two fasting plasma quantities—all had a correlation better than the three-parameter biomarker for the whole population: R²=60-61% (Table 8). The most efficient biomarker appears to be made up of four variables: insulin, lactate, glucose, and triglycerides: R²=79%. Removing insulin only marginally degrades the correlation R²=74%, but removing all the other plasma quantities, including lactate, had a more substantial impact. The equation for the four-variable biomarker is:
GIR=−12.6+0.82G+16.13L+0.076TG−3.42I
where G represents plasma glucose concentration, L represents plasma lactate concentration, TG represents plasma triglycerides concentration and I represents plasma insulin concentration. The four-variable biomarker reflects the complex interactions that regulate insulin sensitivity.

Thus, each subpopulation—subjects with “lactate-associated” and “glucose-associated” insulin resistance—yielded different and better possibilities for assessing insulin resistance when separated from each other. As is apparent from FIG. 23, a few patients, particularly those with very low GIR, appeared in both pools, and reduce the correlations found for all of the potential biomarkers in this study.

TABLE 1
Coefficients and R² values for step-wise regression analysis with preferred prevalence
weightings using “glucose-modulated” patients in this study. Other independent-variable
combinations were tried, with less effective results.

| | Insulin | | | Lactate | | avg glucose - glucose | | Constant | |
|---|---|---|---|---|---|---|---|---|---|
| | (μU/ml) | FFA (mg/dl) | TG (mg/dl) | (mg/dl) | HBA1c (%) | (mg/dl) (mg/dl) | glucose (mg/dl) | (mg/min) | R² |
| Single-variable | | | 0.660 | | | | | 121.3 | 55% |
| correlations | −6.79 | | | | | | | 325.6 | 47% |
| | | 8.23 | | | | | | 54.0 | 42% |
| | | | | 17.74 | | | | 61.0 | 36% |
| | | | | | 13.93 | | | 99.0 | 16% |
| | | | | | | 0.70 | | 170.3 | 15% |
| | | | | | | | 0.50 | 139.5 | 10% |
| Two- | −7.02 | | | | | | 0.57 | 238.3 | 60% |
| | −3.10 | | 0.457 | | | | | 200.0 | 60% |
| | −6.51 | | | | 12.07 | | | 217.4 | 59% |
| | | | 0.525 | 8.76 | | | | 63.3 | 61% |
| Three- | | | 0.289 | 17.70 | | | 0.72 | −96.1 | 74% |
| Four- | −3.42 | | 0.076 | 16.13 | | | 0.82 | −12.6 | 79% |
| All | −3.03 | 1.66 | 0.026 | 16.64 | −141.65 | 3.86 | 4.84 | 260.2 | 79% |

### I. Example 9

### ROC Points for Fasting Biomarkers

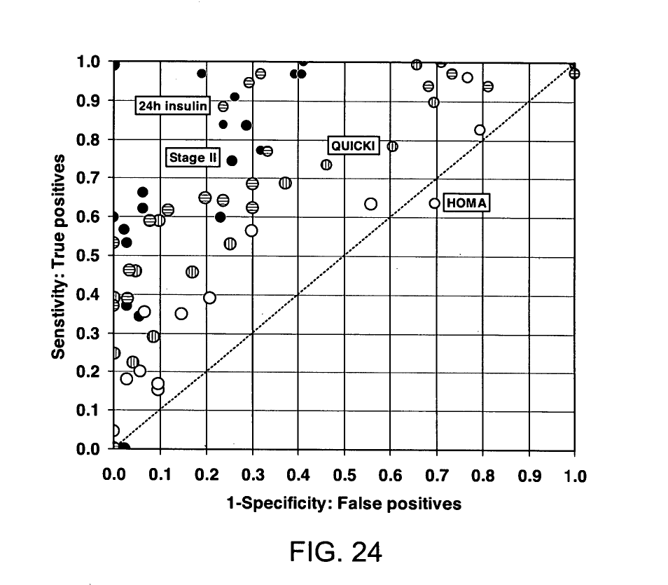

FIG. 24 shows the ROC points for the optimum fasting biomarker developed in this invention, along with the ROC points for a biomarker based on fasting insulin alone. For comparison, the ROC points for QUICKI and HOMA are also shown. As shown, the points do not lie along smooth curves as in the idealized case shown in FIG. 9 because of the discrete nature of the data. In other words, as the threshold moves up the diagonal in FIG. 1, the contribution of a single patient as it “moves” from one quadrant to another is magnified at the extremes of the distribution.

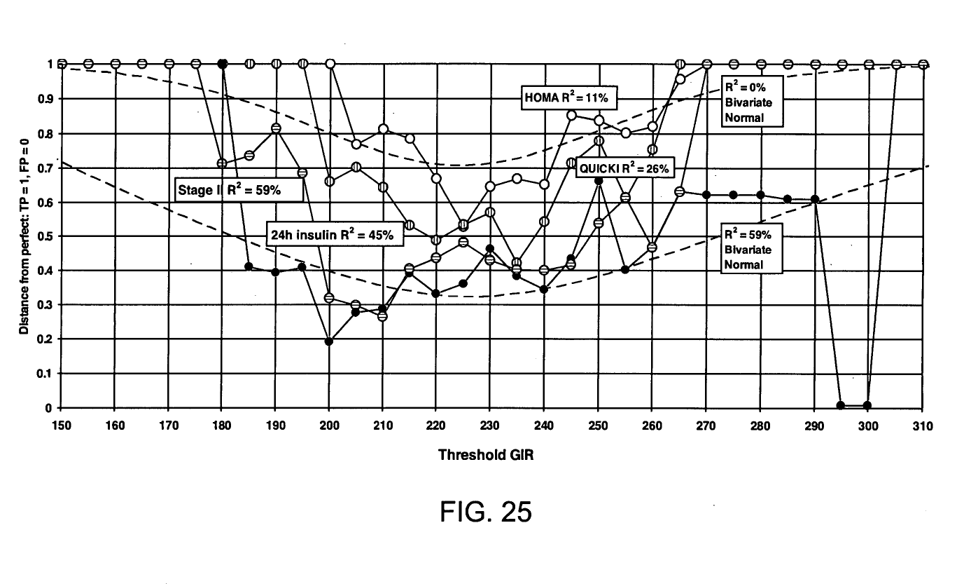

For this reason, a plot of the form of FIG. 5 is easier to read. Such a plot is shown in FIG. 25. Again, at the extremes, the discrete nature of the data is important to note—the curves will take on values of zero or one at the edges of the validity space for the biomarker. The region in the threshold space where these discrete jumps are minimized defines the dynamic range of the biomarker. It is clear that within the dynamic range for HOMA, the ROC points are very similar to the line derived for the idealized, uncorrelated case. This result is to be expected from the low correlation of HOMA with insulin sensitivity as measured by the euglycemic clamp.

In its dynamic range, the optimum biomarker yields a plot similar to that derived from the idealized model with an R² of 59%. The improved predictive value relative to insulin alone is apparent in the broader range of threshold values for which specificity and sensitivity are nontrivial. This increase in dynamic range is characteristic of improved biomarker performance and correlates with increasing R² values.

### J. Example 10

### Postprandial Biomarkers

1. Representing Expected Variability in the Patient Population and Protocol

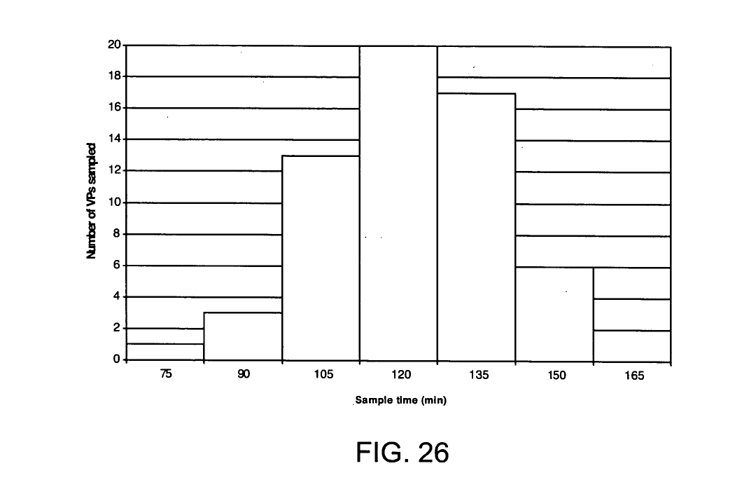

To represent two major sources of variability likely to impact a postprandial biomarker based on a single-time-point measurement, additional variability was represented in the virtual patient population. This variability is meant to represent both individual variability in gastric emptying rates and variability in the time between meal consumption and a plasma sample being collected. This variability was simulated by taking measurements at times randomly distributed around a desired measurement point. The methodology assumed a normal distribution with the fixed point as the mean. A standard deviation of approximately twenty-three minutes was then applied to the distribution, corresponding to ˜95% of the postprandial sampling times falling within forty-five minutes of the desired fixed point. An example distribution around the two-hour fixed point is given in FIG. 26.

To test the robustness of the proposed biomarkers, ten such distributions, specifying different sample times for each virtual patient, were generated for both a two-hour fixed point and a three-hour fixed point. The same set of sample time points was used for each test meal.

2. Test Meals

This stage of the project was originally designed to analyze the effects of two test meals on biomarker robustness. Though these meals had significantly different caloric content and macronutrient compositions, they contained approximately the same amount of carbohydrate. As the analysis progressed, it became clear that larger meals provided more stable biomarkers. The reason for this appears to be that a larger meal provides a relatively constant supply of nutrients from the gut during the sampling window. However, patient compliance for such a large breakfast may be an issue, so a more moderate meal that was sufficient to maintain a relatively constant nutrient supply was included for analysis. Meal compositions are shown in Table 9.

TABLE 9
Sizes and macronutrient characteristics of test meals.

| | Kcal | CHO | Fat | Protein |
|---|---|---|---|---|
| Light | 439 | 78% | 10% | 12% |
| Moderate | 750 | 30% | 50% | 15% |
| Heavy | 1156 | 19% | 58% | 23% |

The light and heavy meals were derived based on the specific ingredients listed in Table 10.

TABLE 10
Meal components

| | |
|---|---|
| Light breakfast | Heavy breakfast |

| | | | |
|---|---|---|---|
| orange juice | 4 oz | omelet | 3 large eggs |
| cereal | 1 cup | cheese | 0.5 cup |
| | | | shredded |
| 1% milk | 1 cup | ham | 0.5 cup |
| | | toast | 2 slices |
| | | bacon | 3 slices |
| | | potatoes | 1 oval patty |
| | | onion | 0.25 cup |
| | | butter | 3 pats |
| | | orange juice | 4 oz |

3. Two-Hour Plasma Measurement

Simulated breakfasts of various sizes were considered by the computer model and all modeled plasma quantities were measured at fifteen-minute intervals for up to five hours after the meal. As discussed, randomly selected samples near measurement times of interest simulated variations in gastric emptying among the virtual patients, a parameter not varied during their development. For practical reasons, measurements around two and three hours were considered appropriate for biomarker analysis.

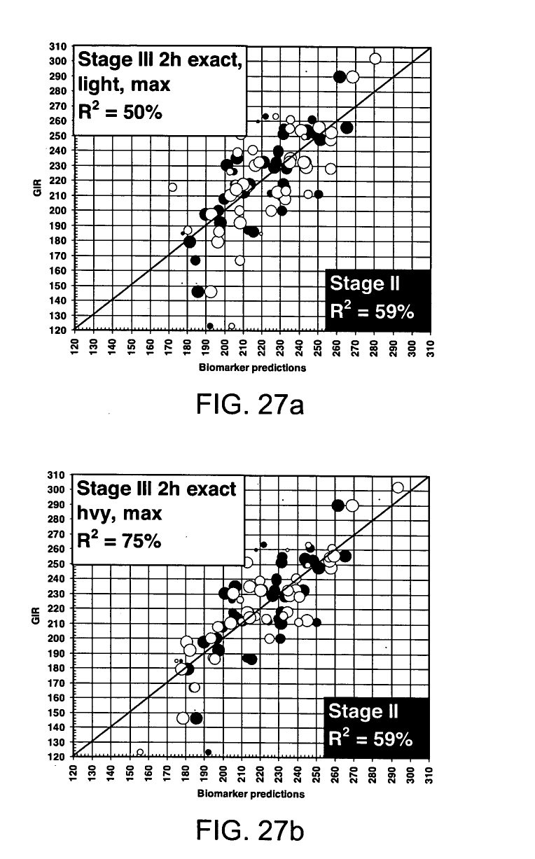

The two-hour light-meal postprandial measurements showed no possibility of improving on the fasting biomarker (FIG. 27). Even without the random perturbations of sample time, and using all possible regressors, the biomarker yielded an R² less than the 59%.

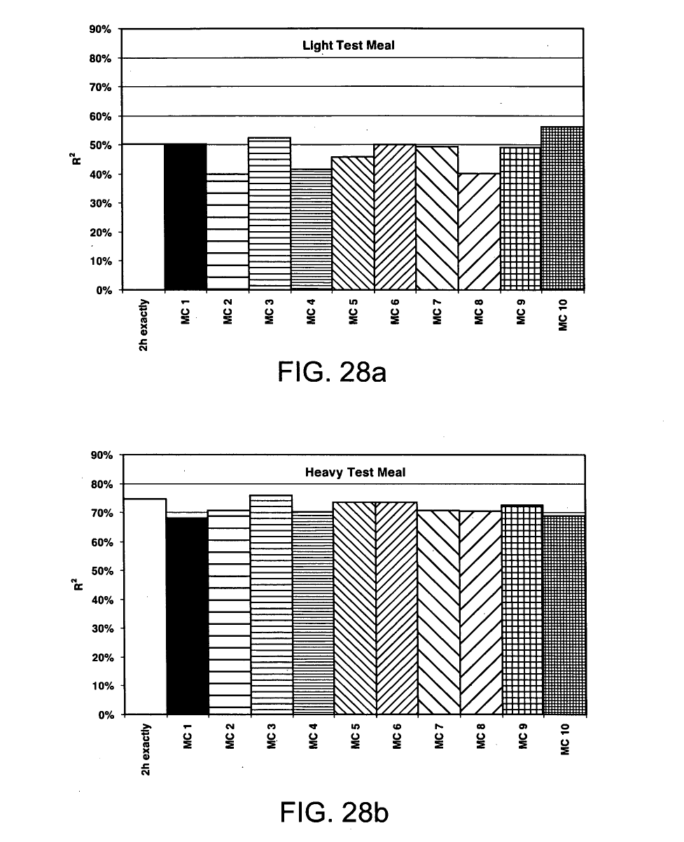

FIG. 28 shows the individual R² values for the exact two-hour fit, and for fits based on ten Monte Carlo simulations for the light and heavy meal, respectively. The light meal correlations show greater variability than those of the heavy meal, and are still too low to be promising. The heavy-meal correlations are more stable, and suggest the possibility of a useful biomarker.

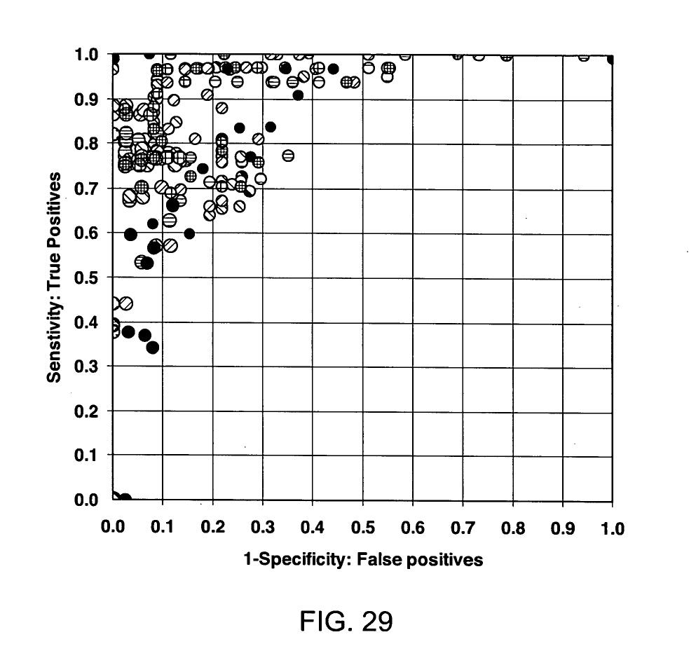

FIG. 29 shows the ROC points for the heavy meal and for all of the random sample-time perturbations, compared to the fasting biomarker. The heavy meal seems, on average, to provide better sensitivity and specificity.

When compared to the average of the ROC points over the ten Monte Carlo realizations, the optimal fasting biomarker is frequently more than two standard deviations above the average, i.e., lies beyond the ˜95% confidence interval. The following simple statistical analysis suggests that these deviations are significant; that is, that the optimal fasting biomarker profile across its dynamic range was likely drawn from a population of less sensitive curves than that of the postprandial biomarker.

4. Statistical Comparison of Fasting and Postprandial Biomarker Performance

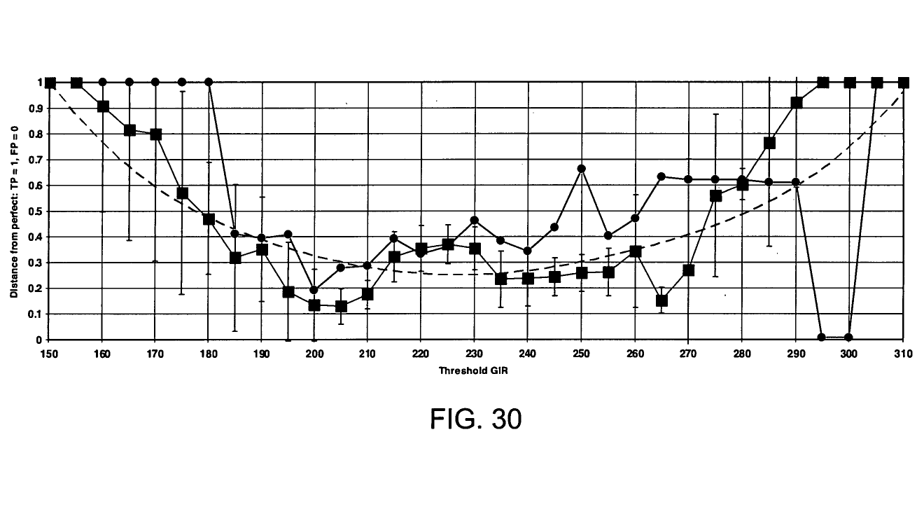

For each threshold in FIG. 30, the probability that a point lies outside the two-standard deviation error bars is less than 5%. There are 21 threshold points in the dynamic range of the postprandial biomarker (between 185-280). Of these, nine of the optimal fasting biomarker points lie above the error bars. To see if this was a statistically viable result, the likelihood that one would observe deviations from the collective means “this large” if the optimal fasting biomarker were drawn from this underlying population of effects were determined. As a first approximation, the method makes the null assumption that each point on the optimal fasting biomarker curve is drawn from an independent population with a mean and standard deviation as given by the Monte Carlo results. Based on this null assumption, the probability of observing a data point more than two standard deviations from the mean is less than 5%. Based on these probabilities, one may estimate the probability of observing nine of twenty-one points being more than two standard deviations away from the mean. That calculation takes advantage of the binomial distribution as follows:

$$( \begin{matrix}N \\ K\end{matrix} ) {{p}^{K} ( 1 - p )}^{N - K} \\ ( \begin{matrix}21 \\ 9\end{matrix} ) {( 0.05 )}^{9} {( 0.95 )}^{12} << 0.01 \qquad Equation 2$$

Where N=number of nontrivial sensitivity-specificity points from fed measurements, i.e., the dynamic range of the biomarker; K=number of optimal fasting biomarker results that lie more than two standard deviations from the mean, i.e., points that are significantly worse than the fed measures; and P=probability that the distance of the fasting biomarker sensitivity and specificity from (0,1) came from same population as the fed value.

Calculating this quantity for the heavy meal sampled at two hours yields a probability of occurrence under the null, a p-value, of less than 5%. Thus, a postprandial biomarker based on a heavy test meal is likely to provide better sensitivity and specificity than a fasting measure. For the light test meal, a similar calculation resulted in a p-value of 0.42, clearly an insignificant benefit.

These results for the heavy meal indicate that a postprandial biomarker, based on the maximum possible set of regressors is useful in predicting the insulin resistance of a given patient population. Before pursuing this analysis to identify an optimal set of biomarker components, consider the utility of a somewhat later measurement of plasma quantities and a more moderate sized test meal.

5. Three-Hour Plasma Measurement

The poor correlation of the light-meal measurements at the two-hour time point were due to the postprandial rise in plasma quantities like glucose and insulin, which peak at about that time. Sampling at three hours seemed a practical alternative.

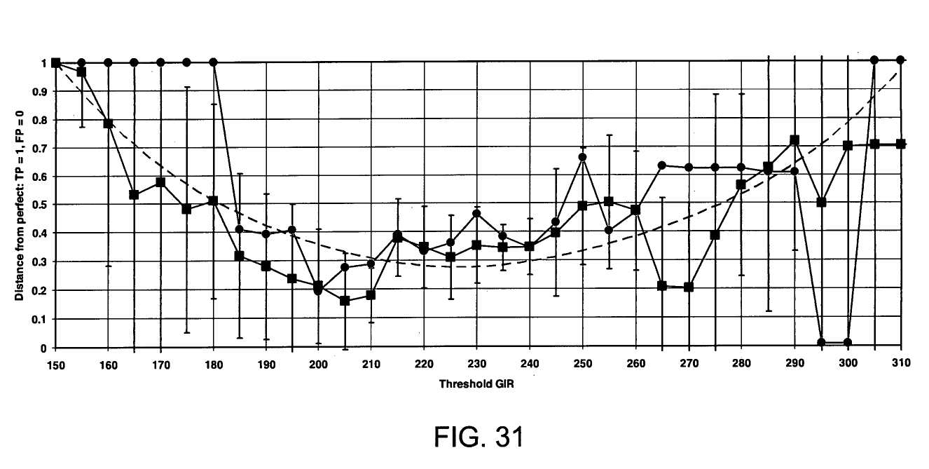

The R² value improved at the three-hour time point for the light meal (FIG. 31). In the dynamic range of the biomarker, four of the twenty-one points were significantly worse for the optimal fasting biomarker, and applying Equation 2 yielded a probability of four points for which there is an improvement of ˜0.02.

The R² value at the three-hour time point for the heavy meal was essentially unchanged and, as for the two-hour point, the probability of the fasting measure consistently performing as well as the fed measure p<0.01.

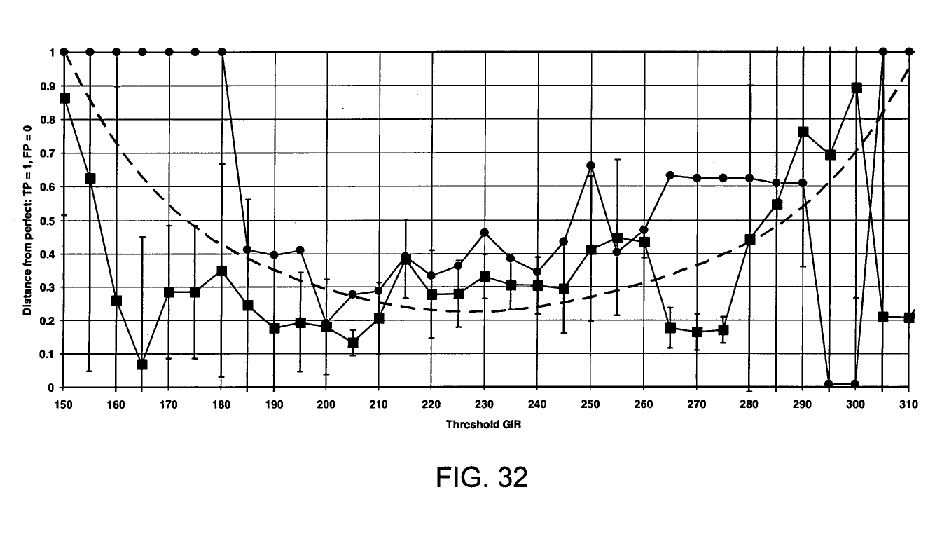

Because the heavy meal might be less practical than a lighter meal for the purposes of a clinical test, a more moderate meal was designed and analyzed. As shown in FIG. 32, at the two-hour time point, it did no better than the heavy meal, but sampling at the three-hour time point gave a result similar to the heavy meal, with a probability of the optimal fasting biomarker doing as well <0.01.

### K. Example 11

### Seeking More Efficient Biomarkers

The two meal sizes and two sample times are two alternative possibilities for a biomarker. The analysis thus far has focused on the best possible biomarker by examining regressions with a maximal set of plasma quantities. This section seeks a minimal set of predictors for the two cases, and the biomarkers presented in this section contain five and seven components for the heavy- and moderate-meals, respectively.

In the following analyses, the ROC points make clearer the significance of eliminating regressors from the biomarker, i.e., although R² might be only modestly affected, the dynamic range can be materially reduced or the better performance of the test vs. the fasting measures becomes inconsistent.

1. Large-Meal, Two-Hour Test

The most efficient biomarker for the large meal sampled at two hours postprandially includes only six plasma quantities, and is defined by the equation:

$$GIR = 776 - 216 * plasma C \text{-} peptide - 14.6 * Hbalc - 0.05 plasma glucose - 417 * plasma glycerol + 4.55 * plasma insulin + 1.80 * plasma lactate$$

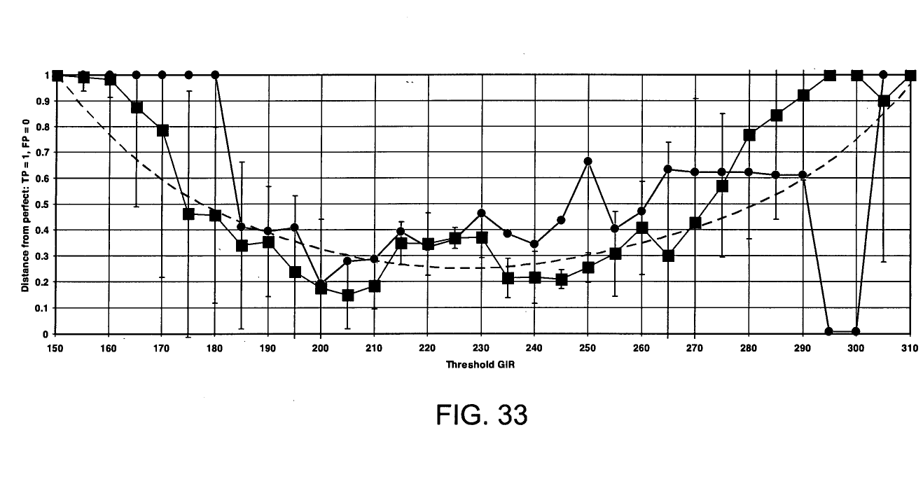

The same analysis outlined above for comparing this biomarker to the optimal fasting biomarker shows that the postprandial biomarker is still significantly more sensitive and specific (FIG. 33) (P<0.05). The quantities included in this biomarker, and the corresponding coefficients for each Monte Carlo run are shown in Table 11. Table 12 shows the corresponding coefficients for the full set of regressors.

TABLE 11
Six regression coefficients for the heavy meal test. Fewer regressors reduced
the dynamic range of the biomarker and the number of points at which the optimal fasting
biomarker was more than two standard deviations from the mean.

| | Coefficients from each Monte Carlo run | | | | | | | | | | |
|---|---|---|---|---|---|---|---|---|---|---|---|
| | exact | 1 | 2 | 3 | 4 | 5 | 6 | 7 | 8 | 9 | 10 |
| plasma | −216 | −15.4 | 22.2 | −79.4 | 74.5 | −41.8 | −41.5 | −75.7 | 59.2 | −78.0 | −39.1 |
| C-pep. | | | | | | | | | | | |
| HBA1c | −14.6 | −32.9 | −30.2 | −41.2 | −42.1 | −43.9 | −40.9 | −22.5 | −35.9 | −26.1 | −32.0 |
| plasma | −0.05 | 0.76 | 0.49 | 0.84 | 0.95 | 0.96 | 0.83 | 0.26 | 0.73 | 0.37 | 0.68 |
| glucose | | | | | | | | | | | |
| plasma | −417 | −143 | −246 | −249 | −97 | −189 | −180 | −293 | −206 | −260 | −180 |
| glycerol | | | | | | | | | | | |
| plasma | 4.55 | −3.07 | −4.83 | −0.93 | −6.41 | −2.16 | −2.28 | −0.76 | −6.05 | −0.99 | −1.92 |
| insulin | | | | | | | | | | | |
| plasma | 1.80 | 1.27 | 3.38 | 0.23 | 2.45 | 1.55 | 2.11 | 2.61 | 3.05 | 0.76 | 1.79 |
| lactate | | | | | | | | | | | |
| Constant | 776 | 548 | 639 | 690 | 537 | 621 | 617 | 650 | 602 | 670 | 565 |

TABLE 12
Maximal set of regression coefficients for heavy meal test

| | Coefficients from each Monte Carlo run | | | | | | | | | | |
|---|---|---|---|---|---|---|---|---|---|---|---|
| | exact | 1 | 2 | 3 | 4 | 5 | 6 | 7 | 8 | 9 | 10 |
| plasma | −224 | −53 | 3 | −145 | −24 | −117 | −114 | −149 | −23 | −79 | −127 |
| C-peptide | | | | | | | | | | | |
| HBA1c | 293 | 190 | 234 | 255 | 175 | 219 | 232 | 258 | 224 | 243 | 209 |
| plasma | 0.150 | 1.240 | 2.613 | 2.153 | 3.920 | 3.202 | 3.791 | 2.814 | 2.944 | 2.217 | 3.246 |
| chylo. TG | | | | | | | | | | | |
| plasma FFA | −0.633 | −1.161 | 2.228 | −1.639 | 1.905 | −0.217 | 1.878 | −0.291 | 0.609 | 2.393 | 0.791 |
| plasma | −0.133 | −0.059 | −0.190 | −0.191 | 0.058 | −0.075 | −0.182 | −0.285 | −0.049 | −0.192 | 0.002 |
| glucagon | | | | | | | | | | | |
| plasma | −8.68 | −5.54 | −6.96 | −7.53 | −5.23 | −6.50 | −6.94 | −7.69 | −6.67 | −7.23 | −6.22 |
| glucose | | | | | | | | | | | |
| plasma | −418 | −151 | −315 | −267 | −170 | −243 | −292 | −343 | −298 | −319 | −262 |
| glycerol | | | | | | | | | | | |
| plasma | 4.76 | −1.80 | −4.21 | 1.01 | −2.76 | 0.40 | 0.18 | 1.71 | −3.28 | −0.99 | 1.09 |
| insulin | | | | | | | | | | | |
| plasma | 1.98 | 1.33 | 2.96 | 0.37 | 0.95 | 0.92 | 1.46 | 2.72 | 2.93 | 0.86 | 1.48 |
| lactate | | | | | | | | | | | |
| plasma TG | 0.000 | −0.082 | −0.194 | −0.205 | −0.304 | −0.258 | −0.274 | −0.191 | −0.204 | −0.143 | −0.233 |
| (mg/dl) | | | | | | | | | | | |
| glucose | 8.64 | 6.17 | 7.58 | 8.28 | 6.14 | 7.42 | 7.63 | 7.82 | 7.23 | 7.71 | 6.71 |
| deviation from | | | | | | | | | | | |
| HBA1c wtd | | | | | | | | | | | |
| avg glucose | | | | | | | | | | | |
| constant | 139 | 91 | 111 | 121 | 84 | 104 | 110 | 123 | 107 | 115 | 99 |

2. Moderate-Meal, Three-Hour Test

The coefficients for maximal biomarker using all eleven regressors are given in Table 4 for the fixed three-hours sampling time and for each of the Monte Carlo simulations. The ROC points shown in FIG. 24 were calculated using these eleven plasma quantities. Employing the same methods as above, the most efficient biomarker for this case was identified (Table 5).

The most efficient biomarker for this case is given by the following equation:

$$GIR = 323 + 2.4 * plasmaFFA + 0.33 * plasma glucagon - 0.149 * plasma glucose - 2.46 * plasma insulin - 1.17 * plasma lactate + 0.092 * plasma TG + 0.503 * ( glucose deviation from avg glucose )$$

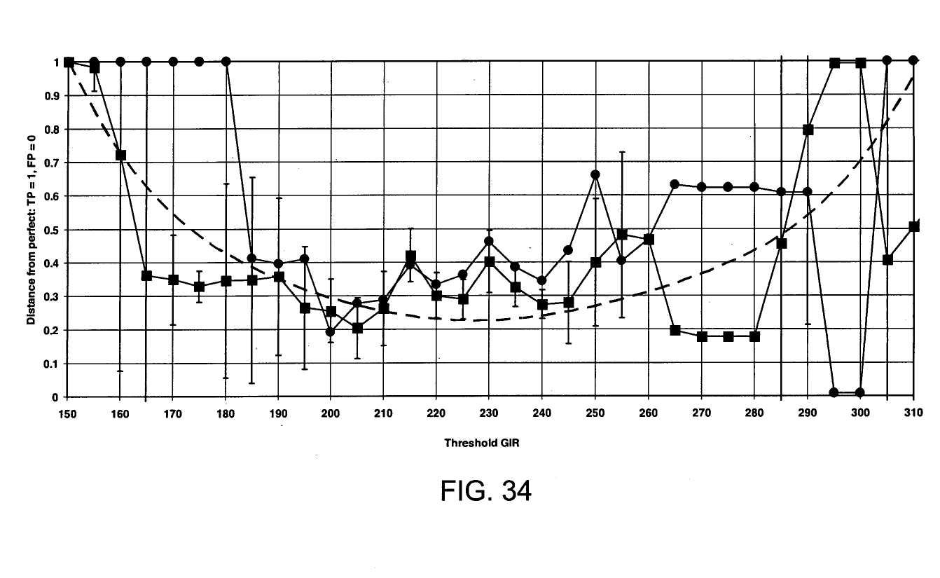

FIG. 34 shows the distance metric for the ROC when the seven plasma quantities were included. The number of points better than the optimal fasting biomarker is still significant.

TABLE 13
Seven regression coefficients for moderate meal test. Fewer regressors
reduced the dynamic range and the number of points that were better than the optimal
fasting biomarker.

| | Coefficients from each Monte Carlo run | | | | | | | | | | |
|---|---|---|---|---|---|---|---|---|---|---|---|
| | exact | 1 | 2 | 3 | 4 | 5 | 6 | 7 | 8 | 9 | 10 |
| plasma | 2.43 | 2.30 | 0.43 | 3.30 | 3.15 | 2.87 | 0.87 | 3.41 | 4.50 | 2.65 | 4.32 |
| FFA | | | | | | | | | | | |
| plasma | 0.332 | 0.399 | 0.157 | 0.634 | 0.170 | 0.347 | 0.178 | 0.412 | 0.693 | 0.290 | 0.719 |
| glucagon | | | | | | | | | | | |
| plasma | −0.149 | −0.116 | −0.122 | −0.274 | −0.192 | −0.197 | −0.087 | −0.116 | −0.172 | −0.126 | −0.152 |
| glucose | | | | | | | | | | | |
| plasma | −2.46 | −2.24 | −2.41 | −2.08 | −2.65 | −2.14 | −2.20 | −2.23 | −1.83 | −2.38 | −1.70 |
| insulin | | | | | | | | | | | |
| plasma | −1.17 | −1.38 | −1.09 | −1.82 | −1.29 | −1.43 | −1.41 | −1.64 | −2.28 | −1.62 | −2.26 |
| lactate | | | | | | | | | | | |
| plasma | 0.092 | 0.068 | 0.088 | 0.066 | 0.057 | 0.108 | 0.048 | 0.068 | 0.053 | 0.090 | 0.096 |
| TG | | | | | | | | | | | |
| (mg/dl) | | | | | | | | | | | |
| glucose | 0.503 | 0.539 | 0.819 | 0.665 | 0.861 | 0.810 | 1.059 | 0.733 | 0.652 | 0.479 | 0.396 |
| deviation | | | | | | | | | | | |
| from | | | | | | | | | | | |
| HBA1c | | | | | | | | | | | |
| wtd avg | | | | | | | | | | | |
| glucose | | | | | | | | | | | |
| constant | 323 | 300 | 335 | 298 | 352 | 301 | 310 | 284 | 248 | 326 | 239 |

TABLE 14
Maximal set of regression coefficients for moderate meal test.

| | Coefficients from each Monte Carlo run | | | | | | | | | | |
|---|---|---|---|---|---|---|---|---|---|---|---|
| | exact | 1 | 2 | 3 | 4 | 5 | 6 | 7 | 8 | 9 | 10 |
| plasma | −28.8 | −33.5 | −45.5 | −34.2 | −55.2 | −40.8 | −66.8 | −41.5 | −32.6 | −9.2 | −48.4 |
| C-peptide | | | | | | | | | | | |
| HBA1c | 210 | 196 | 271 | 193 | 268 | 214 | 206 | 253 | 198 | 189 | 227 |
| plasma | −2.47 | −1.72 | −5.58 | −2.11 | −2.50 | −2.68 | −4.65 | −4.18 | −3.72 | −1.09 | −2.72 |
| chylo. TG | | | | | | | | | | | |
| plasma FFA | 3.91 | 3.09 | −1.10 | 4.96 | 5.13 | 4.77 | −0.06 | 6.46 | 6.04 | 4.60 | 6.72 |
| plasma | 0.228 | 0.245 | −0.199 | 0.522 | 0.202 | 0.319 | 0.215 | 0.384 | 0.517 | 0.196 | 0.505 |
| glucagon | | | | | | | | | | | |
| plasma | −6.19 | −5.75 | −7.91 | −5.86 | −7.94 | −6.39 | −6.11 | −7.45 | −5.88 | −5.49 | −6.76 |
| glucose | | | | | | | | | | | |
| plasma | −280 | −231 | 40 | −239 | −461 | −309 | −119 | −489 | −298 | −253 | −404 |
| glycerol | | | | | | | | | | | |
| plasma | −1.597 | −1.330 | −1.031 | −1.098 | −0.920 | −0.996 | −0.288 | −1.244 | −0.998 | −2.259 | −0.437 |
| insulin | | | | | | | | | | | |
| plasma | −1.09 | −0.87 | −0.62 | −1.25 | −0.79 | −0.81 | −1.16 | −1.39 | −2.07 | −1.46 | −1.68 |
| lactate | | | | | | | | | | | |
| plasma TG | 0.218 | 0.159 | 0.347 | 0.208 | 0.187 | 0.261 | 0.309 | 0.309 | 0.304 | 0.159 | 0.235 |
| (mg/dl) | | | | | | | | | | | |
| glucose | 6.41 | 5.82 | 8.06 | 5.88 | 8.03 | 6.45 | 6.20 | 7.60 | 5.75 | 5.73 | 6.54 |
| deviation | | | | | | | | | | | |
| from HBA1c | | | | | | | | | | | |
| wtd avg glucose | | | | | | | | | | | |
| constant | 100.1 | 93.5 | 125.1 | 91.2 | 128.1 | 101.4 | 97.6 | 120.1 | 94.2 | 90.1 | 107.6 |

Various modifications and variations of the described biomarkers and methods of the invention will be apparent to those of skill in the art without departing from the scope and spirit of the invention. Although the invention has been described in connection with specific preferred embodiments, it should be understood that the invention as claimed should not be unduly limited so such specific embodiments. Indeed, various modifications of the described modes for carrying out the invention that are obvious to those skilled in the art are intended to be within the scope of the following claims.

We claim:

1. A biomarker for assessing insulin resistance of a subject comprising:
(a) a plasma insulin concentration;
(b) a plasma glucose concentration; and
(c) a plasma lactate concentration
wherein the subject fasts prior to measurement of the plasma insulin, glucose and lactate concentrations.

2. The biomarker of claim 1, having the formula:
GIR=126−5.05I+13.3L+0.370G
wherein I represents plasma insulin concentration, L represents plasma lactate concentration and G represents plasma glucose concentration.

3. A biomarker for assessing insulin resistance of a subject comprising:
(a) a plasma insulin concentration;
(b) a plasma glycosylated hemoglobin concentration; and
(c) a plasma lactate concentration
wherein the subject fasts prior to measurement of the plasma insulin, glucose and lactate concentrations.

4. The biomarker of claim 3, having the formula:
GIR=100−4.74I+12.5L+10.2HbA1c
wherein I represents plasma insulin concentration, L represents plasma lactate concentration and HcA1c represents plasma glycosylated hemoglobin concentration.

5. A biomarker for assessing insulin resistance of a lactate-associated subject comprising:
(a) a plasma insulin concentration; and
(b) a plasma lactate concentration
wherein the lactate-associated subject fasts before measurement of the plasma insulin and lactate concentrations.

6. A biomarker for assessing insulin resistance of a glucose-associated subject comprising:
(a) a plasma insulin concentration;
(b) a plasma glucose concentration;
(c) a plasma lactate concentration; and
(d) a plasma triglyceride concentration
wherein the glucose-associated subject fasts prior to measurement of the plasma insulin, glucose, lactate and triglyceride concentrations.

7. A biomarker for assessing insulin resistance in a subject comprising:
(a) a plasma insulin concentration;
(b) a plasma glucose concentration;
(c) a plasma lactate concentration;
(d) a plasma HbA1c concentration;
(e) a plasma glycerol concentration; and
(f) a plasma C-peptide concentration
wherein the plasma insulin, glucose, lactate, HbA1c, glycerol and C-peptide concentrations are measured about two hours to about four hours after the subject consumes a heavy meal.

8. The biomarker of claim 7 having the formula:
$$GIR = 776 - 216 * plasma C \text{-} peptide - 14.6 * Hbalc - 0.05 plasma glucose - 417 * plasma glycerol + 4.55 * plasma insulin + 1.80 * plasma lactate .$$
wherein the plasma insulin, glucose, lactate, HbA1c, glycerol and C-peptide concentrations are measured about two hours to about four hours after the subject consumes a heavy meal.

9. A biomarker for assessing insulin resistance in a subject comprising:
(a) a plasma insulin concentration;
(b) a plasma glucose concentration;
(c) a plasma lactate concentration;
(d) a plasma glucagon concentration;
(e) a plasma free fatty acid concentration;
(f) a plasma tri glyceride concentration; and
(g) a deviation of measured plasma glucose concentration from average plasma glucose concentration
wherein the plasma insulin, glucose, lactate, glucagon, triglyceride and free fatty acid concentrations are measured about three hours after the subject consumes a heavy meal.

10. The biomarker of claim 9, having the formula:
$$GIR = 323 + 2.4 * plasmaFFA + 0.33 * plasma glucagon - 0.149 * plasma glucose - 2.46 * plasma insulin - 1.17 * plasma lactate + 0.092 * plasma TG + 0.503 * ( glucose deviation from avg glucose ) .$$
wherein the plasma insulin, glucose, lactate, glucagon, triglyceride and free fatty acid concentrations are measured about three hours after the subject consumes a heavy meal.

11. A method of assessing insulin resistance in a fasting subject comprising:
(a) measuring a plasma insulin concentration in the fasting subject;
(b) measuring a plasma glucose concentration in the fasting subject;
(c) measuring a plasma lactate concentration in the fasting subject;
(d) calculating a predicted euglycemic hyperinsulinemic clamp glucose infusion rate (GIR); and
(e) diagnosing the subject as being insulin resistant when the predicted GIR has a value of less than about 6 mg/kg-min.

12. A method of assessing insulin resistance in a lactate-associated fasting subject comprising:
(a) measuring a plasma insulin concentration in the lactate-associated fasting subject;
(b) measuring a plasma lactate concentration in the lactate-associated fasting subject;
(c) calculating a predicted euglycemic hyperinsulinemic clamp glucose infusion rate (GIR); and
(d) diagnosing the subject as being insulin resistant when the predicted GIR is less than about 6 mg/kg-min

13. A method of assessing insulin resistance in a glucose-associated fasting subject comprising:
(a) measuring a plasma insulin concentration in the glucose-associated fasting subject;
(b) measuring a plasma glucose concentration in the glucose-associated fasting subject;
(c) measuring a plasma lactate concentration in the glucose-associated fasting subject;
(d) measuring a plasma triglyceride concentration in the glucose-associated fasting subject;
(e) calculating a predicted euglycemic hyperinsulinemic clamp glucose infusion rate (GIR); and
(f) diagnosing the subject as being insulin resistant when the predicted GIR is less than about 6 mg/kg-min.

14. A method of assessing insulin resistance in a subject comprising:
(a) measuring a plasma insulin concentration in the subject about two to about four hours after a heavy meal;
(b) measuring a plasma glucose concentration in the subject about two to about four hours after a heavy meal;
(c) measuring a plasma lactate concentration in the subject about two to about four hours after a heavy meal;
(d) measuring a plasma glycosylated hemoglobin (HbA1c) concentration about two to about three hours after a heavy meal;
(e) measuring a plasma glycerol concentration in the subject about two to about four hours after a heavy meal;
(f) measuring a plasma C-peptide concentration in the subject about two to about four hours after a heavy meal;
(g) calculating a euglycemic hyperinsulinemic clamp glucose infusion rate (GIR) using the formula:
$$GIR = 776 - 216 * plasma C \text{-} peptide - 14.6 * Hbalc - 0.05 plasma glucose - 417 * plasma glycerol + 4.55 * plasma insulin + 1.80 * plasma lactate . ; and$$
(h) diagnosing the patient as being insulin resistant when the predicted GIR is less than about 6 mg/kg-min.

15. A method of assessing insulin resistance in a subject comprising:
(a) measuring a plasma insulin concentration in the subject about three hours after a moderate meal;
(b) measuring a plasma glucose concentration in the subject about three hours after a moderate meal;
(c) measuring a plasma lactate concentration in the subject about three hours after a moderate meal;
(d) measuring a plasma glucagon concentration about three hours after a moderate meal;
(e) measuring a plasma free fatty acid concentration in the subject about three hours after a moderate meal;
(f) measuring a deviation of measured plasma glucose concentration from average plasma glucose concentration in the subject about three hours after a moderate meal; and
(g) calculating a predicted euglycemic hyperinsulinemic glucose infusion rate (GIR) using the formula:
$$GIR = 323 + 2.4 * plasmaFFA + 0.33 * plasma glucagon - 0.149 * plasma glucose - 2.46 * plasma insulin - 1.17 * plasma lactate + 0.092 * plasma TG + 0.503 * ( glucose deviation from avg glucose )$$
and
(h) diagnosing the subject as being insulin resistant when the predicted GIR is less than about 6 mg/kg-min.

16. A kit for evaluating insulin resistance in a subject, the kit comprising:
a device for obtaining a blood sample from the subject;
a reagent for measuring a concentration of glucose (G) in the blood sample;
a reagent for measuring a concentration of lactate (L) in the blood sample;
a reagent for measuring a concentration of insulin (I) in the blood sample; and
instructions for use.

17. The kit of claim 16, wherein the kit indicates insulin resistance when the formula
GIR=126−5.05I+13.3L+0.370G
provides a value of GIR of less than about 6.

18. A kit for evaluating insulin resistance in a subject, the kit comprising:
a device for obtaining a blood sample from the subject;
a reagent for measuring a concentration of glycosylated hemoglobin (HbA1c) in the blood sample;
a reagent for measuring a concentration of lactate (L) in the blood sample;
a reagent for measuring a concentration of insulin (I) in the blood sample;
and instructions for use.

19. The kit of claim 18, wherein the kit indicates insulin resistance with the formula:
GIR=100−4.74I+12.5L+10.2HbA1c
provides a value of GIR of less than about 6.

20. The kit of claim 19, wherein the kit indicates insulin resistance when GIR is less than about 5.
